(19) United States
(12) Patent Application Publication
(10) Pub. No.: US 2019/0365784 A1
(43) Pub. Date: Dec. 5, 2019

(54) USE OF BRASSINOSTEROID ANALOGS FOR THE TREATMENT OF DERMAL DISORDERS BY SELECTIVELY MODULATING LIVER X RECEPTORS (LXR) AND DERMAL DISEASE TREATMENT BY BRASSINOSTEROID ANALOGS ACTING AS SELECTIVE LIVER X RECEPTOR (LXR) MODULATORS

(71) Applicants: Raúl Enrique MASSONE, Buenos Aires (AR); CONSEJO NACIONAL DE INVESTIGACIONES CIENTÍFICAS Y TÉCNICAS (CONICET), Buenos Aires (AR); INSTITUTO MASSONE S.A., Buenos Aires (AR)

(72) Inventors: José GROISMAN, Buenos Aires (AR); Felipe INSERRA, Buenos Aires (AR); Elena MARTINEZ VIVOT, Buenos Aires (AR); Emilio SOJO, Buenos Aires (AR)

(21) Appl. No.: 16/067,354

(22) PCT Filed: Dec. 29, 2016
(86) PCT No.: PCT/IB2016/058082

§ 371 Date: Jun. 29, 2018

Related U.S. Application Data
(60) Provisional application No. 62/272,900, filed on Dec. 30, 2015.

(51) Int. Cl. A61K 31/575; A61P 17/06

(52) U.S. Cl. CPC A61K 31/575; A61P 17/06

(57) ABSTRACT

Brassinosteroid analogs used for the treatment of dermal disorders or conditions. One embodiment is the topical application of at least one brassinosteroid analog for the treatment of psoriasis in a mammal, the brassinosteroid analogs being of general formula (a). (a) (I) where, R₁, R₂, and R3 are selected from H, HO—, linear or branched C1-C4 alkyl, R₅—O—, HCOO—, R₅—COO—, —OOC—R₆—COO—, p-toluene sulphonate, phosphate, tartrate, maleate, sulphate, fluorine, chlorine, bromine, iodine and methanesulphonate, R₄ and R₅ are selected from H and linear or branched C1-C4 alkyl, R₆ is —(CH2)n- wherein n equals to 1, 2 or 3, and can be a single or double bond. A method of therapeutic treatment for psoriasis, skin aging, rosacea, dermatitis, burns, skin cancer and malignancies, and pigmentary derangements, by element of administration of brassinosteroid analogs of general formula (a).

### FIELD OF THE INVENTION

The present invention relates to the use of brassinosteroid analogs for the treatment of dermal disorders or conditions. One embodiment is the topical application of at least one brassinosteroid analog described herein for the treatment of psoriasis in a mammal.

The present invention describes the use of brassinosteroid analogs of general formula (a).

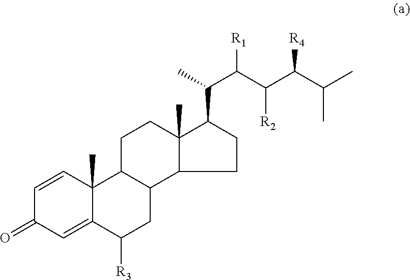

wherein,

R₁, R₂, and R₃ are selected from H, HO—, linear or branched C1-C4 alkyl, R₅—O—, HCOO—, R₅—COO—, —OOC—R₆—COO—, p-toluene sulphonate, phosphate, tartrate, maleate, sulphate, fluorine, chlorine, bromine, iodine and methanesulphonate,

R₄ and R₅ are selected from H and linear or branched C1-C4 alkyl,

R₆ is —(CH2)n- wherein n equals to 1, 2 or 3, and

[symbol] can be a single or double bond

An embodiment of the present invention describes a method of therapeutic treatment for psoriasis, photoaging, rosacea and UV induced skin cancer, by means of administration of brassinosteroids of general formula (a).

Preferable compounds of general formula (a) are selected from the following:
I (22S,23S)-22,23-dihydroxystigmast-4-en-3-one
II (22S,23S)-22,23-dihydroxystigmasta-1,4-dien-3-one
III (22S,23S)-6α-fluoro-22,23-dihydroxystigmast-4-en-3-one
IV (22S,23S)-6β-fluoro-22,23-dihydroxystigmast-4-en-3-one
V (22S,23S)-6α-fluoro-22,23-dihydroxystigmasta-1,4-dien-3-one
VI (22S,23S)-6β-fluoro-22,23-dihydroxystigmasta-1,4-dien-3-one

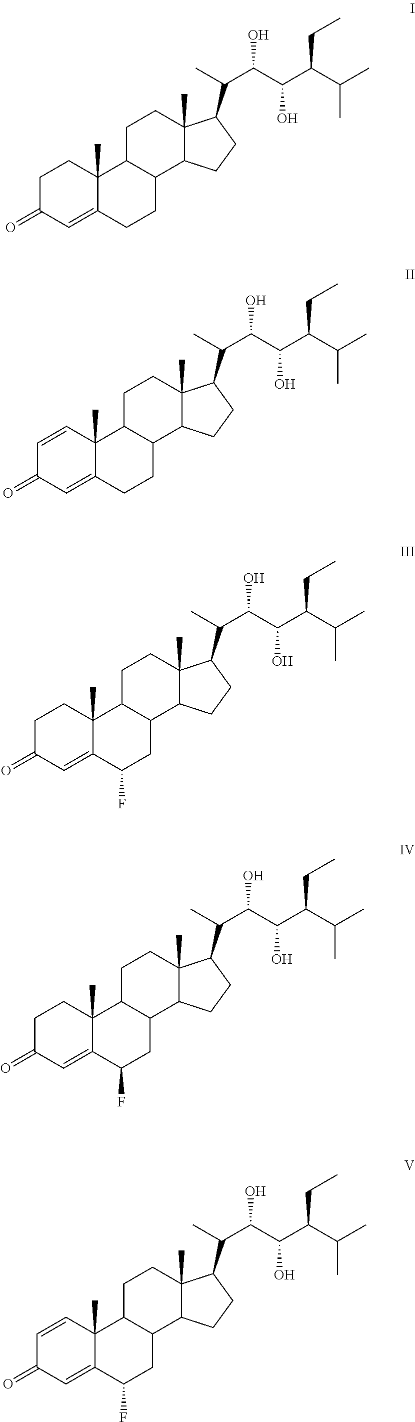 [SMILES: CC[C@@H](C(C)C)[C@H](O)[C@@H](O)C(C)[C@H]1CCC2C3CCC4=CC(=O)CC[C@]4(C)C3CC[C@@]21C ; CC[C@@H](C(C)C)[C@H](O)[C@@H](O)C(C)[C@H]1CCC2C3CCC4=CC(=O)C=C[C@]4(C)C3CC[C@@]21C ; CC[C@@H](C(C)C)[C@H](O)[C@@H](O)C(C)[C@H]1CCC2C3C[C@H](F)C4=CC(=O)CC[C@]4(C)C3CC[C@@]21C ; CC[C@@H](C(C)C)[C@H](O)[C@@H](O)C(C)[C@H]1CCC2C3C[C@@H](F)C4=CC(=O)CC[C@]4(C)C3CC[C@@]21C ; CC[C@@H](C(C)C)[C@H](O)[C@@H](O)C(C)[C@H]1CCC2C3C[C@H](F)C4=CC(=O)C=C[C@]4(C)C3CC[C@@]21C]

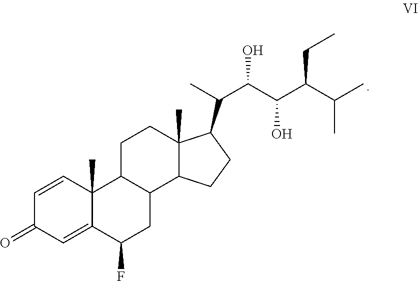 [SMILES: CC[C@@H](C(C)C)[C@H](O)[C@@H](O)C(C)[C@H]1CCC2C3C[C@@H](F)C4=CC(=O)C=C[C@]4(C)C3CC[C@@]21C]

### BACKGROUND OF THE INVENTION

Brassinosteroids (BRs) are a group of naturally occurring polyhydroxy steroidal plant hormones that control plant growth and development. BRs are found at low levels in pollen, seeds, and young vegetative tissues throughout the plant kingdom. Due to their very low concentration in plants, to study their biological activities it is necessary to obtain BRs by chemical synthesis. In order to investigate BR derivatives for their potential medical uses, our group has synthetized 30 BR analogs.

The U.S. Pat. No. 8,431,554 B2 discloses Brassinosteriods, as the present invention, which have anti-inflammatory and antiviral activity. In pharmaceutical compositions, the compounds are useful in ophthalmic pharmaceuticals for treatment of diseases caused by adenovirus, such as epidemic keratoconjunctivitis, and herpes simplex type 1, such as herpetic stromal keratitis.

The U.S. Pat. No. 9,187,518 B2 discloses a method of treating a solid tumor in a mammal by inhibiting angiogenesis, including administering to the mammal, which has a solid tumor selected from the group consisting of breast carcinoma, lung carcinoma, prostate carcinoma, colon carcinoma, ovarian carcinoma, neuroblastoma, central nervous system tumor, multiform glioblastoma and melanoma; with a composition including brassinosteroids as the present invention.

The U.S. Pat. No. 8,987,318 B2 discloses that the skin disorders that present an altered or dysfunctional epidermal barrier include inflammation to mucous membranes (such as cheilitis, chapped lips, nasal irritation, vulvovaginitis; eczematous dermnatitides, such as atopic and seborreheic dermatitis, allergic or irritant contact dermatitis, eczema craquelee, photoallergic dermatitis, phototoxic dermatitis, phytophotodermatitis, radiation dermatitis, and stasis dermatitis; ulcers and erosion resulting from trauma, burns, bullous disorders, or ischemia of the skin or mucous membranes; several forms of ichthyoses; epidermolysis bullosae; hypertrophic scars and keloids and cutaneous changes of intrinsic aging and photoaging, and the like.

The publication of Michelini et al., “Anti-herpetic and anti-inflammatory activities of two new synthetic 22,23-dihydroxylated stigmastane derivatives” Journal of Steroid Biochemistry & Molecular Biology 111 (2008), 111-116, shows the brassinosteroid (BRs) compounds I and II of the present invention.

The publication of Michelini F M, Bueno C A, Molinari A M, Galigniana M D, Galagovsky L R, Alché L E, Ramirez J A. “Synthetic stigmastanes with dual antiherpetic and immunomodulating activities inhibit ERK and Akt signaling pathways without binding to glucocorticoid receptors”. Biochimica et Biophysica Acta 1860 (2016) 129-139, shows the brassinosteroid (BRs) compounds I, II, III, IV, V and VI used in the present invention.

### SUMMARY OF THE INVENTION

The present invention refers to a method of treatment of skin diseases comprising administering to a patient in need thereof of a composition that comprises brassinosteroid analogs of general formula (a)

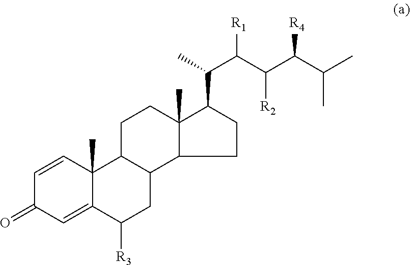

wherein,

R₁, R₂, and R₃ are selected from H, HO—, linear or branched C1-C4 alkyl, R₅—O—, HCOO—, R₅—COO—, —OOC—R₆—COO—, p-toluene sulphonate, phosphate, tartrate, maleate, sulphate, fluorine, chlorine, bromine, iodine and methanesulphonate,

R₄ and R₅ are selected from H and linear or branched C1-C4 alkyl,

R₆ is —(CH₂)ₙ— wherein n equals to 1, 2 or 3, and

[symbol] can be a single or double bond,

and a pharmacologically acceptable excipient.
The method of treatment of skin diseases indicated the brassinosteroid analogs are selected from the group comprising:

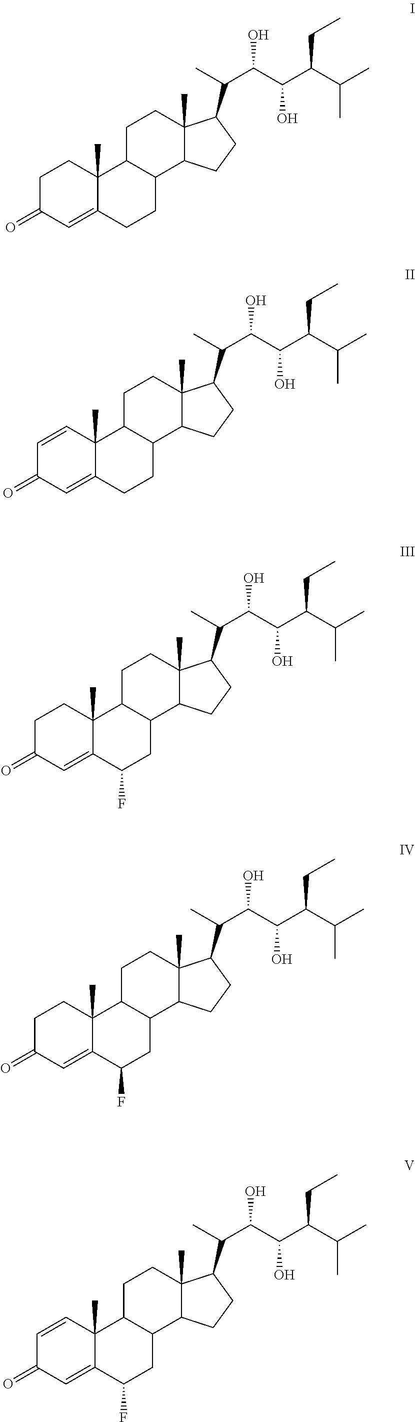 [SMILES: CC[C@@H](C(C)C)[C@H](O)[C@@H](O)C(C)[C@H]1CCC2C3CCC4=CC(=O)CC[C@]4(C)C3CC[C@@]21C ; CC[C@@H](C(C)C)[C@H](O)[C@@H](O)C(C)[C@H]1CCC2C3CCC4=CC(=O)C=C[C@]4(C)C3CC[C@@]21C ; CC[C@@H](C(C)C)[C@H](O)[C@@H](O)C(C)[C@H]1CCC2C3C[C@H](F)C4=CC(=O)CC[C@]4(C)C3CC[C@@]21C ; CC[C@@H](C(C)C)[C@H](O)[C@@H](O)C(C)[C@H]1CCC2C3C[C@@H](F)C4=CC(=O)CC[C@]4(C)C3CC[C@@]21C ; CC[C@@H](C(C)C)[C@H](O)[C@@H](O)C(C)[C@H]1CCC2C3C[C@H](F)C4=CC(=O)C=C[C@]4(C)C3CC[C@@]21C]

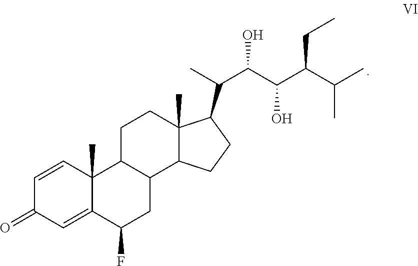 [SMILES: CC[C@@H](C(C)C)[C@H](O)[C@@H](O)C(C)[C@H]1CCC2C3C[C@@H](F)C4=CC(=O)C=C[C@]4(C)C3CC[C@@]21C]

In said method of treatment, the skin disease is selected from the group comprising psoriasis, skin aging, rosacea, dermatitis, burns, skin cancer and malignancies, and pigmentary derangements, wherein the skin aging includes chronological aging and UV-induced aging and wherein the pigmentary derangements include vitiligo.

In the method of treatment of skin diseases according to present invention, the composition is a systemic or topical composition.

In an embodiment of the invention, in the method of treatment of skin diseases the composition further comprises corticosteroids.

In other embodiment, the composition is separately or sequentially administered with corticosteroids.

The corticosteroids are selected but not limited to the group comprising hydrocortisone, triamcinolone, fluocinonide, betamethasone dipropionate, clobetasol, fluocinolone acetonide, prednisone, prenisolone, and dexamethasone.

In a different embodiment, present invention is also directed to a composition comprising brassinosteroid analogs of general formula (a)

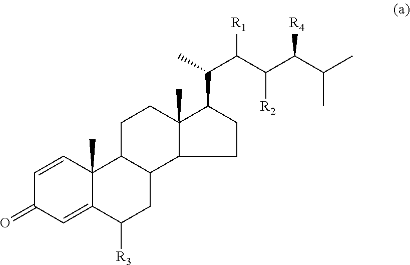

wherein,

R₁, R₂, and R₃ are selected from H, HO—, linear or branched C1-C4 alkyl, R₅—O—, HCOO—, R₅—COO—, —OOC—R₆—COO—, p-toluene sulphonate, phosphate, tartrate, maleate, sulphate, fluorine, chlorine, bromine, iodine and methanesulphonate,

R₄ and R₅ are selected from H and linear or branched C1-C4 alkyl,

R₆ is —(CH₂)ₙ— wherein n equals to 1, 2 or 3, and

[symbol] can be a single or double bond, and a pharmacologically acceptable excipient for use treatment of skin diseases.

In the composition for use treatment of skin diseases indicated, the brassinosteroid analogs are selected from the group comprising:

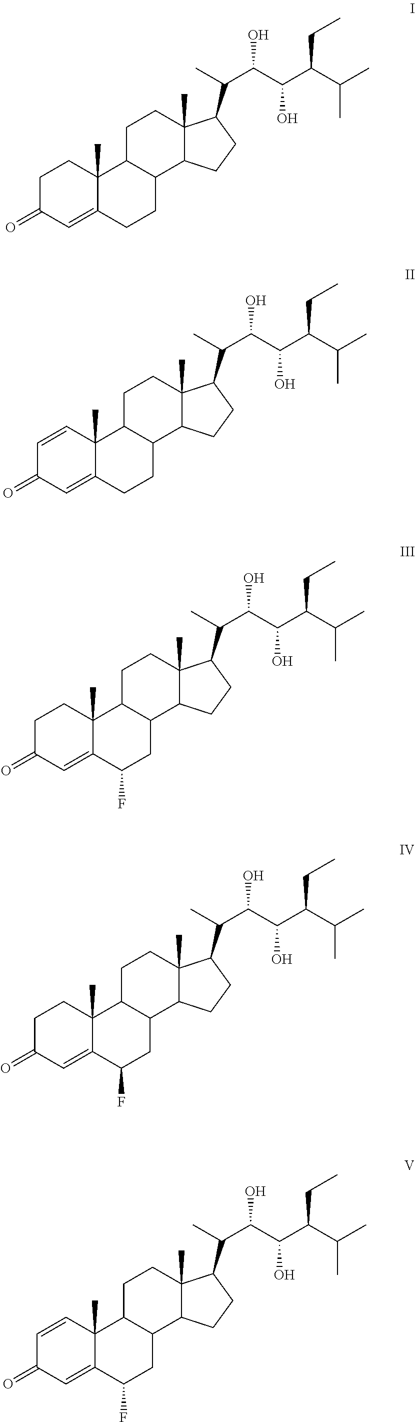 [SMILES: CC[C@@H](C(C)C)[C@H](O)[C@@H](O)C(C)[C@H]1CCC2C3CCC4=CC(=O)CC[C@]4(C)C3CC[C@@]21C ; CC[C@@H](C(C)C)[C@H](O)[C@@H](O)C(C)[C@H]1CCC2C3CCC4=CC(=O)C=C[C@]4(C)C3CC[C@@]21C ; CC[C@@H](C(C)C)[C@H](O)[C@@H](O)C(C)[C@H]1CCC2C3C[C@H](F)C4=CC(=O)CC[C@]4(C)C3CC[C@@]21C ; CC[C@@H](C(C)C)[C@H](O)[C@@H](O)C(C)[C@H]1CCC2C3C[C@@H](F)C4=CC(=O)CC[C@]4(C)C3CC[C@@]21C ; CC[C@@H](C(C)C)[C@H](O)[C@@H](O)C(C)[C@H]1CCC2C3C[C@H](F)C4=CC(=O)C=C[C@]4(C)C3CC[C@@]21C]

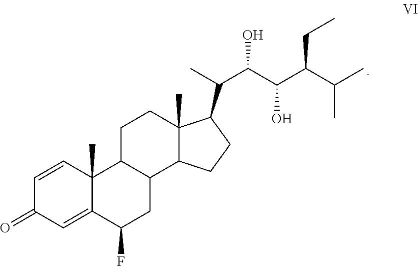 [SMILES: CC[C@@H](C(C)C)[C@H](O)[C@@H](O)C(C)[C@H]1CCC2C3C[C@@H](F)C4=CC(=O)C=C[C@]4(C)C3CC[C@@]21C]

In said composition for use in the treatment of skin diseases, the skin disease is selected from the group comprising psoriasis, skin aging, rosacea, dermatitis, burns, skin cancer and malignancies, and pigmentary derangements, wherein the skin aging includes chronological aging and UV-induced aging and wherein the pigmentary derangements include vitiligo.

The composition for use in the treatment of skin diseases of present invention can be a systemic or topical composition.

In an embodiment of the invention, the composition for use in the treatment of skin diseases further comprises corticosteroids.

In other embodiment, the composition for use in the treatment of skin diseases is separately or sequentially administered with corticosteroids.

The corticosteroids are selected but not limited to the group comprising hydrocortisone, triamcinolone, fluocinonide, betamethasone dipropionate, clobetasol, fluocinolone acetonide, prednisone, prenisolone, dexamethasone.

### DESCRIPTION OF THE DRAWINGS

FIG. 1: A and B: are graphs that show that Compounds I, II, IV and VI induce gene transcription by activating LXR-alpha.

A. After transfection with plasmids carrying LXR-alpha, the retinoid X receptor (RXR), a LXR response element-luciferase reporter (LRE-LUC) and RSV-LacZ (as a transfection control), HEK293T cells were incubated with either vehicle [0.1% (v/v) DMSO], or GW3965 (1 uM), or Compounds I, II, III, IV, V and VI (10 uM each). The results are presented as fold-induction of LXR-alpha driven luciferase activity versus the commercial LXR-alpha/beta agonist GW3965. *p<0.05 vs. DMSO; § p<0.05 vs. GW3965, not different from DMSO; B. BHK cells were transfected with plasmids carrying LXR-alpha, the retinoid X receptor (RXR) and a LXR response element-luciferase reporter (LRE-LUC). After transfection the cells were incubated with either vehicle (DMSO), or GW3965 (1 uM), or Compounds I (COMP I) or II (COMP II) (1 uM, 3 uM and 10 uM), COMP I and COMP II stimulated LXR-alpha-driven gene transcription [(LXRE)-luciferase activity] within the same concentration range as GW3965. (LXRE)-luciferase activity was measured in cell lysates. LXRE-driven luciferase activity is expressed as relative light units (RLU) *p=0.0286 vs. DMSO; Mann-Whitney.

FIG. 2: are graphs that show that Compound I reduces back skin erythema, but has no effect on scaling.

FIG. 3: is a graph that shows Back skin thickness after 9 days of Compound I or clobetasol treatment.

None of the assayed Compound 1's doses prevented back skin thickening, whereas clobetasol treatment inhibited IMQ-induced skin thickening in the back.

FIG. 4: is a graph that shows Group Mean Scores of dorsal skin in psoriatic mice treated with Compound I or clobetasol.

Histopathological analysis of back skin samples from vehicle-treated psoriatic controls were generally the most severely affected Samples from 0.05% (w/v) Compound I-treated mice generally had slightly lower severity of microscopic alterations, relative to vehicle-treated psoriatic controls. In samples from 0.10% (w/v) Compound I-treated mice the severity of microscopic alterations was lower than in samples from vehicle-treated psoriatic controls and 0.05% (w/v) Compound I-treated mice. The lowest severity of microscopic alterations was associated to clobetasol treatment.

FIG. 5: is a graph that shows Group global response scores of dorsal skin in psoriatic mice treated with Compound I or clobetasol.

In clobetasol-treated psoriatic mice, the global response score of dorsal skin was ˜42% lower than in vehicle-treated psoriatic mice, whereas in Compound I (0.1%)-treated mice it was ˜26% lower vs. vehicle-treated animals.

FIG. 6: is a graph that shows Body weight change in mice after 9 days of topical Compound I or clobetasol treatments.

Psoriatic mice topically treated with clobetasol for 9-days showed a 15% reduction of body weight compared with vehicle-treated mice. In Compound I-treated animal body weight was 8% lower than in naïve mice, and there was no change relative to vehicle-treated animals.

FIG. 7: is a graph that shows Low mice spleen weight after 9 days of topical clobetasol treatment.

Psoriatic mice topically treated with clobetasol for 9-days showed a 62.6% reduction of spleen weight compared with the vehicle-treated psoriatic mice. In Compound I-treated animals spleen weight was 20% higher than in naïve mice, and showed no change relative to vehicle-treated animals.

FIG. 8: is a graph that shows TNF-alpha expression in UV irradiated HaCaT cells exposed to Compound I or Compound II or GW3965.

HaCaT cells were pre-incubated for 1 h in the presence of 10 uM of either Compound I or Compound II, or GW3065. After aspiration of the culture medium, the cells were exposed to UV radiation (254 nm, 15 J/m²; immediately after the cultured medium was replenished, and after 6 h or 24 h the cells were collected for RNA isolation and purification, followed by retrotranscription and real time amplification of CDNA. TNF-alpha expression was normalized to GAPDH expression. Twenty-four hours after UV irradiation, TNF-alpha expression was significantly lower in cells incubated with Compounds I or II or with GW3965 versus DMSO-treated cells [*p=0.032 vs. DMSO (UV, 24 h), Mann Whitney]. In GW3965-treated cells TNF-alpha expression was significantly higher than in Comp I-treated cells [‡0.045 vs. Comp I (UV, 24 h), Mann Whitney.

FIG. 9: are graphs that show IL-8 and ABCA1 expression in UV-irradiated HaCaT cells exposed to Compound I or Compound II or GW3965.

HaCaT cells were pre-incubated for 1 h in the presence of 10 uM of either Compound I or Compound II, or GW3065. After aspiration of the culture medium, the cells were exposed to UV radiation (254 nm, 15 J/m²; immediately after the culture medium was replenished, and after 6 h or 24 h the cells were collected for RNA isolation and purification, followed by retrotranscription and real time amplification of cDNA. IL-8 and ABCA1 expressions were normalized to GAPDH expression. A. Twenty four hours after UV irradiation, IL-8 expression was significantly lower in cells incubated with Compounds I or II [*p=0.034 vs. DMSO (UV, 24 h), Mann Whitney] or GW3965 versos DMSO-treated cells [‡p=0.047 vs. DMSO (UV, 24 h), Mann Whitney]. B. In all groups, ABCA1 expression at 24 h after UV irradiation was higher than in DMSO-treated cells (*p=0.038, Mann Whitney; FIG. 9, B); however, the induction of ABAC1 expression in GW3965-treated cells was stronger than in Comp I- or Comp II-treated cells, in both non-irradiated HaCaT (†p=0.024, Mann Whitney) and 24 h after UV exposure (‡p=0.001, Mann Whitney).

FIG. 10: are graphs that show LXR-alpha and LXR-beta expressions in UV irradiated HaCaT cells exposed to Compound I or Compound II or GW3965.

HaCaT cells were pre-incubated for 1 h in the presence of 10 uM of either compound I or Compound II, or GW3065. After aspiration of the culture medium, the cells were exposed to UV radiation (254 nm, 15 J/m²; immediately after the culture medium was replenished, and after 6 h or 24 h the cells were collected for RNA isolation and purification, followed by retrotranscription and real time amplification of cDNA. LXR-alpha and LXR-beta expressions were normalized to GAPDH expression. A. Twenty four hours after UV-irradiation, LXR-alpha expression was higher in HaCaT incubated in the presence of DMSO compared to non-irradiated cells and GW3965-treated cells (‡p=0.036, Mann Whitney). However, both compound I and Compound II stimulated LXR-alpha expression even further [*p=0.021 vs. DMSO (UV, 24 h) and GW3965 (UV, 24 h), Mann Whitney]. B. LXR-beta expression responded to UV irradiation and to incubation with either Compound I, or Compound II, or GW3965, or DMSO in the same direction as LXR-alpha expression, although the magnitude of the changes were less marked [‡p=0.044 vs. non-irradiated (UV, 24 h) and GW3965-treated Ha-Cat (UV, 24 h), Mann Whitney]. Compound I stimulated LXR-beta expression even further [*p=0.032 vs. DMSO (UV, 24 h) and GW3965 (UV, 24 h) Mann Whitney]. In compound II treated cells, LXR-beta Induction at 24 h after UV irradiation was more modest than that observed for Compound I [†p=0.040 vs. GW3965 treated cells (UV, 24 h)].

FIG. 11: is a graph that shows Fatty acid synthase (FAS) expression in HL-60 cells treated with GW3965 or Compounds I or II for 6 h.

Data were obtained from 3 independent assays conducted in duplicate, they were normalized against GAPDH expression and are expressed as average ±SE. *p<0.05 vs. DMSO; ***p<0.001 vs. DMSO.

FIG. 12: are graphs that show ABCA1 (A.) and ABCG1 (B.) expressions in HL-60 cells treated with GW3965 or Compounds I or II for 6.

Data were obtained from 3 independent assays conducted in duplicate, they were normalized against GAPDH expression and are expressed as average ±SE. ***p<0.001 vs. DMSO.

FIG. 13: are graphs that show FASN (A.) and SRBEP1 (B.) expressions in HepG2 cells treated with GW3965 or Compounds I or II for 6 h.

Data were obtained from 3 independent assays conducted in duplicate, they were normalized against GAPDH expression and are expressed as average ±SE. **p<0.001 vs. all others.

FIG. 14: are graphs that show ABCG1 (A.) and ABCA1 (B.) expressions in HepG2 cells treated with GW3965 or Compounds I or II for 6 h.

Data were obtained from 3 independent assays conducted in duplicate, they were normalized against GAPDH expression and are expressed as average ±SE. *p<0.05 vs. Comp I 1, 3 and 10 uM; **p<0.001 vs. all others; ***p<0.01 vs. DMSO, Comp I 1 and 3 uM, Comp II 1 and 3 uM.

FIG. 15: are graphs that show LXR-alpha (A.) and LXR-beta (B.) expressions in HepG2 cells cells treated with GW965 or Compound I or II for 6 hours.

Data were obtained from 3 independent assays conducted in duplicate, they were normalized against GAPDH expression and are expressed as average ±SE. ****p<0.001 vs. all others; **p<0.05 vs. DMSO, Comp I 1 and 3 uM, Comp II 1, 3 and 10 uM.

FIG. 16: are graphs that show Total cholesterol (A), HDL-cholesterol (B) and triglyceride (C) plasmatic contents in C57BI mice treated with T0901317 or Compound I for 5 days. Male C57BI mice received daily for 5 days intraperitoneal injections containing either 10 mg Compound I/kg, or 5 mg T0901317/kg, or vehicle (90% (v/v) DMSO in apyrogenic sterile water. IN T0901317-treated mice total cholesterol, HDL-cholesterol and triglyceride plasmatic contents were significantly higher (_{↑}50%, 50%, and >2-times, respectively) than in compound I- or Vehicle-treated mice. *p<0.01; **p<0.001; ***p>0.05 vs. DMSO and Comp I.

FIG. 17: is a graph that shows Liver weight in C57BI mice treated with T0901317 or Compound I for 5 days.

Male C57BI mice received daily for 5 days intraperitoneal injections containing either 10 mg Compound I/kg, or 5 mg T0901317/kg, or vehicle (90% (v/v) DMSO in apyrogenic sterile water. In T0901317-treated mice the liver weights were ˜23% higher than in Compound I or DMSO treated-mice. ***p<0.001

FIG. 18: shows pictures of Gross liver morphology in C57BI mice treated with T0901317 or Compound I for 5 days. Five day treatment with T0901317, but not with Compound I, induces liver steatosis, as indicated by the increase of liver weight (FIG. 17) and paler color in mice that received T0901317.

### DETAILED DESCRIPTION OF THE INVENTION

It is well-known that LXRs are ligand activated transcription factors that belong to the nuclear receptor (NR) superfamily. Two LXRs have been described, LXR-alpha and LXR-beta, both of which form heterodimers with the retinoid X receptor (RXR). The LXR/RXR heterodimer binds to LXR response elements (LXREs), whose main characteristic is the presence of direct repeats of the consensus sequence AGGTCA, separated by four nucleotides. The endogenous LXR ligands are oxysterols (oxidized cholesterol derivatives), and some bile acids; synthetic ligands include GW3965 and T0901317 that do not discriminate between LXR-alpha and LXR-beta. LXR-alpha expression is mainly found in the liver, kidney, gall bladder, lung, testes, spleen, intestine, adipose tissue, and skin, whereas LXR-beta is ubiquitously expressed. LXR-alpha and LXR-beta amino acid sequences are about 77% identical. LXR-alpha and LXR-beta are present in all layers of the epidermis, and their activation is associated with the improvement of altered epidermal barrier formation, and the reductions of cell hyperproliferation and skin inflammation, while stimulating keratinocyte differentiation. In addition, activation of LXR modulates signaling pathways that participate in the pathophysiology of skin aging.

Psoriasis is a skin condition characterized by hyperproliferation and abnormal differentiation of epidermal keratinocytes, epidermal barrier dysfunction, lymphocyte infiltration, and dermal endothelial vascular changes.

Keratinocyte differentiation is a sequential process that leads to the formation of the stratum corneum composed of terminally differentiated keratinocytes known as corneocytes. Corneocytes possess a cornified envelope that results from the extensive cross-linking of various proteins on the inner plasma membrane by the enzyme transglutaminase-1. In addition, corneocytes provide the necessary scaffold for the organization of extracellular lipids into lamellar bodies, the organelles that deliver lipids to the stratum corneum. LXR activation not only induces the expression of many genes required for keratinocyte differentiation, but also increases the expression of the proteins needed for the formation of the cornified envelope.

Two of the crucial functions of the stratum corneum are the prevention of both a) excessive water loss through the epidermis and b) permeation into epidermal and dermal cells of environmental compounds that can induce an immune response. The lipid composition of the stratum corneum—comprising mainly cholesterol, free fatty acids and ceramides—is a key feature for the epidermal barrier function.

Of note, alteration of epidermal barrier integrity or function results in epidermal hyperplasia, seemingly because barrier deficiency signals to the nucleated epidermal cells to proliferate in order to achieve barrier restoration. These signals may be the main event that conducts to hyperproliferative skin disorders, such as psoriasis, ichthyosis, acanthosis nigricans, eczema, atopic dermatitis, rosacea, and non-melanoma skin cancers.

### Barrier Function, Cholesterol and LXR

Abundant cholesterol content in keratinocytes is required for epidermal barrier function; therefore, the latter depends closely on the regulation of cholesterol homeostasis. LXRs are key regulators of cholesterol homeostasis. Circulating cholesterol levels are regulated by balancing dietary cholesterol absorption, intracellular synthesis, and excess cholesterol elimination from peripheral tissues. When intracellular cholesterol concentration increases, cells metabolize this compound to oxysterols which—upon binding LXRs—activate LXR-mediated transcription to induce cholesterol efflux and diminish cholesterol synthesis and influx. LXRs control reverse cholesterol transport (mobilization of peripheral cholesterol towards the liver) by inducing the expression of the ATP-binding cassette (ABC) family of sterol transporters, such as ABCG1 and ABCA1, which promote cellular cholesterol efflux to be transported by high density lipoproteins (HDL) to the liver for degradation/excretion. In contrast, low-density lipoprotein (LDL) and very low density-lipoprotein (VLDL) are responsible for transporting cholesterol from the liver to peripheral tissues when it is needed for cell membrane synthesis, nervous tissue signaling, or as a substrate for steroid hormone, vitamin D or bile acid synthesis.

Plasma LDL-cholesterol is taken up by cellular LDL receptors (LDLR), whose function is regulated by sterol regulatory element binding proteins (SREBPs). LXR activation down-regulates this pathway by increasing the expression of IDOL (inducible degrader of LDLR) which prompts LDLR degradation.

SREBPs are transcription factors that regulate the biosynthesis of cholesterol, fatty acid, and triglyceride, by controlling the expression of genes involved in lipogenesis and lipid uptake.

SREBP-2 upregulates the expression of the LDL receptor and the majority of cholesterol biosynthetic enzymes, whereas SREBP-1c promotes the transcription of genes for fatty acid synthesis—including acetyl-CoA carboxylase and fatty acid synthase-, and triglyceride synthesis.

Triglyceride-loaded VLDLs transport lipids from hepatic to adipose and other peripheral tissues. LXR activation also stimulates triglyceride transport to adipose tissue by increasing the expression of proteins involved in lipid transfer—such as phospholipid transfer protein and cholesterol ester transfer protein—in addition to lipoprotein lipase.

It has been shown that topical treatment with certain LXR agonists stimulates the maturation and differentiation of a functional stratum corneum, including a functional epidermal barrier.

In addition, oxysterols stimulate keratinocyte differentiation and improve epidermal barrier function. Accordingly, cistrome mapping in normal human epidermal keratinocytes identified 2035 LXR-beta-RXR-alpha binding sites that contained 4794 LXR response elements, and revealed the presence of consensus heterodimer binding regions in genes involved in keratinocyte lipid transport/synthesis and terminal differentiation. Epidermal barrier formation and maintenance is dependent on lipid synthesis, lipid transport, and keratinocyte differentiation, and derangement of their functions predisposes to skin inflammation.

The improvement of the epidermal barrier function by LXR agonists is mediated by at least two mechanisms: the stimulation of lipid synthesis and keratinocyte differentiation.

### LXR and Cell Proliferation

In addition to other functions, cholesterol can regulate cell proliferation and embryonic development as a result of the key role it plays as a component of cell membranes. Hence, to allow for membrane synthesis, cellular proliferation and differentiation, cholesterol metabolism and the expression of lipogenic genes must be finely coordinated. In this context, cholesterol starvation leads to cell cycle arrest, and inhibition of cholesterol synthesis diminishes cell growth which is reversed by adding cholesterol. This evidence points to a critical role of cholesterol homeostasis in cellular proliferation, underscoring the crucial participation of LXR-alpha and -beta since they are responsible for the regulation of cholesterol metabolism. In this setting, agonist-mediated LXR activation limits the proliferation of various types of cells by interfering with cell cycle control and survival signals. The mechanism seems to involve LXR-dependent cholesterol mobilization, which can modify the structure of lipid rafts, since cholesterol largely contributes to their structure and function. Lipid rafts are cholesterol-rich plasma membrane domains that function as crucial signaling hubs that regulate a variety of cell functions, including apoptotic pathways.

In addition, treatment of normal mice with topical LXR agonists reduced epidermal thickness and keratinocyte proliferation and augmented cell death; also, in hyperproliferative epidermis, oxysterol treatment reestablished epidermal homeostasis as evidenced by stimulated keratinocyte differentiation and decreased hyperproliferation.

LXR agonist's ability to oppose epidermal hyperplasia points to these compounds as beneficial agents for the treatment of skin conditions associated with keratinocyte hyperproliferation and/or disturbed differentiation. In this context, the present invention provides methods and compositions for improving epidermal lipid synthesis and reducing several signs of psoriasis.

### UV, Photoaging and LXR

Solar UV radiation damages the skin eventually leading to photoaging, i.e., premature skin aging accompanied by the appearance of wrinkles, irregular pigmentation and loss of skin firmness and hydration. Of note, solar UV radiation has been shown to reduce the barrier function of human skin, resulting from UV effects on intercellular components of the stratum corneum (such as corneo-desmosomes and intercellular lipids) that alter cell cohesion and mechanical integrity.

Photoaging affects epidermal keratinocytes, dermal fibroblasts, and infiltrating neutrophils. In skin keratinocytes, UV radiation activates activator protein 1 (AP-1) and nuclear factor-kappa B (NF-kappaB), resulting in increased matrix metalloproteinases (MMPs) and cytokine expression.

LXR agonists were shown to protect against the effects of UV irradiation in normal human keratinocytes and in a model of photoaging in mice. These results were supported by results in cultured human epidermal keratinocytes and skin cell preparations, showing that LXR activators stimulates the expression of genes for fatty acid and ceramide synthesis in keratinocytes, and for cholesterol binding proteins and lipid transporters in skin cells; also they increase the expression of keratinocyte differentiation markers and reduce metalloproteinases and cytokine expressions in UV-irradiated epidermal keratinocytes and TNF-alpha activated dermal fibroblasts.

### Rosacea—Potential for LXR Activation

Rosacea is a common skin condition that mainly affects the face. The early pathophysiology of this condition includes an increase in the innate immune response to certain stimuli and neuroimmune/neurovascular imbalances. UV exposure seems to act as a main external trigger that activates innate immunity and/or neurogenic responses.

Skin infiltration by inflammatory cells, angiogenesis, altered extracellular matrix composition, increased IL-8 (a chemoattractant cytokine that also stimulates angiogenesis) and VEGF-alpha expressions, and derangement of the skin permeability barrier function have been found in rosacea. Whereas some of the above pathogenic mechanisms are targeted by available therapies, others remain as potential objectives for the development of new therapeutic compounds.

There is still some controversy over whether chronic UV-induced skin damage is a primary pathogenic agent for rosacea, or—alternatively—the skin alterations associated to photo-damage (loss of perivascular integrity, extracellular matrix degradation, erythema, development of telangiectasia) amplify analogous structural changes related to rosacea.

At present, there is no cure for rosacea, but treatments are available only to help control the symptoms.

We propose that the compounds of the invention would improve rosacea symptoms, due to their ability to improve barrier function and modulate cytokine content through LXR activation.

### LXR Activation: Adverse Effects

LXRs control reverse cholesterol transport (mobilization of peripheral cholesterol towards the liver) by inducing the expression of the ATP-binding cassette (ABC) family of sterol transporters, such as ABCG1 and ABCA1. Also, LXR activation in the liver induces de novo lipogenesis (that is, triglyceride production) mediated by the induction of SREBP1c, acetyl CoA carboxylase (ACC), stearoyl-CoA desaturase 1 (SCD1) and fatty acid synthase (FAS), which as a whole lead to increased liver and plasma triglyceride (TG) contents. Endogenous and synthetic LXR agonists strongly induce ABCA1 expression, thereby increasing HDL cholesterol plasma levels and preventing atherosclerosis in rodents. However, systemic administration of synthetic LXR agonists increases the expression of lipogenic genes in the liver, both directly and by activating SREBP1C, resulting in unwanted hepatic steatosis and hypertriglyceridemia. In consequence, successful design of LXR agonists requires compounds that are able to provide the beneficial actions of LXR activation while circumventing their deleterious secondary effects.

### The Pathogenesis of Psoriasis

Psoriasis is a chronic disease that affects ˜1-3% of Caucasian subjects worldwide, and is considered the most common human autoimmune disease. It is a clinically heterogeneous condition, whose most frequent clinical type is plaque psoriasis (psoriasis vulgaris) that accounts for 90% of psoriasis cases and is mainly characterized by the recurrent emergence of focal to coalescing erythematous cutaneous plaques and consistent scaling. Lesions may be localized or widespread; the widespread type includes erythrodermic, guttate, and generalized pustular psoriasis. There are various levels of severity among psoriasis patients. According to the European consensus, the severity of plaque psoriasis is graded into mild and moderate to severe disease. Mild disease refers to the involvement of 10% of the body surface area (BSA), a psoriasis area and severity index (PASI)≤10 and a dermatology life quality index (DLQI)≤10; whereas moderate to severe psoriasis defines the involvement of >10% BSA or PASI>10 and DLQI>10. Nearly 80% of psoriasis patients present the mild form of the disease.

The distinctive features of psoriatic lesions include epidermal cell hyperplasia, disruption of the epidermal barrier function, leukocyte infiltration and a profusely developed vascular network. Concerning the role of LXRs in psoriasis, in cultured primary keratinocytes from normal subjects, LXR-alpha gene knockdown simulates the genomic profile found in biopsies from human psoriatic skin lesions. This suggests that restoring LXR-alpha expression/function within a psoriatic lesion may contribute to reverse gene expression transition, leading to the conversion from psoriatic to symptomless skin.

### Current Psoriasis Therapy

The aim of psoriasis therapy is to diminish the severity and the extent of the disease so as to promote the patient's well-being. The European consensus recommended treatment of mild psoriasis with topical agents and moderate to severe psoriasis with phototherapy or systemic treatments. However, patients receiving systemic therapy will probably continue to need some topical agents. The benefits of topical therapy include symptomatic relief, minimization of the systemic medication dosage, and may also provide psychological catharsis for some patients. Although a range of treatment options exists, effective psoriasis treatment is elusive and there is a continuing need for improvement.

Current psoriasis therapy includes topical treatments, light therapy and systemic medications, including tars, emollients, retinoids, dithranol, keratolytics, calcineurin inhibitors, vitamin D analogues, and corticosteroids.

Among these, topical corticosteroids are the backbone of anti-psoriasis pharmacologic arsenal for mild to moderate psoriasis.

Nonetheless, topical corticosteroid use is limited by the accompanying adverse effects, including skin atrophy, telangiectases and/or striae, and secondary systemic effects (see below). Skin atrophy probably results from the anti-AP1 activity of glucocorticoids, because keratinocyte differentiation depends on the expression of genes coding for a variety of proteins under the transcriptional control of AP1. To counteract these unwanted consequences, discontinuous or pulse corticosteroid dosing, as well as reducing topical corticosteroid dosage as a result of combining with other topical compounds, are suggested to increase treatment efficacy and corticosteroid safety for longer use. According to the UK classification, the potency of corticosteroids falls into four groups: mild, moderately potent, potent and very potent.

Mild corticosteroids are prescribed for treatment of the face, axillary areas and groin, as well as for infants and children, while for all other areas mid- and higher potency corticosteroids are usually recommended in adults. In the case of persistent psoriatic lesions of the palms, soles and/or scalp, superpotent corticosteroids are generally advised. Also, potent and superpotent corticosteroids are frequently used initially to accelerate the reduction of the symptoms, with the precaution of strict patient surveillance and treatment restricted to no more than two weeks.

### Systemic Adverse Effects of Topical Corticosteroids

Corticosteroids promote an ample variety of adverse effects that result from their wide array of interactions with specific and non-specific cellular targets. In general the adverse effects of systemic corticosteroids are stronger than with topical treatment; however, topical corticosteroids are associated to unwanted systemic reactions given that the barrier function is damaged in psoriatic skin, thereby facilitating corticosteroid penetration without regard to their potency. In addition, the characteristic blood vessel dilation of psoriatic skin augments the chances of topical corticosteroids gaining the systemic circulation; consequently, if an ample body surface is affected by psoriasis and topical corticosteroid treatment is prolonged, a high concentration of circulating corticosteroid is more likely to occur increasing the risk of adverse systemic effects. High potency corticosteroids are associated to a higher chance of unwanted systemic actions. Among the latter, since corticosteroids depress immune function, opportunistic bacterial, fungal or viral infections are more likely to settle in. The list of adverse systemic glucocorticoid effects also include glaucoma and cataracts (that can be established due to corticosteroid-mediated mineralocorticoid receptor activation), dyslipidemia, coagulopathy, cardiovascular impairment, and worsening of pre-existing diabetes due to the metabolic actions of corticosteroids, muscle atrophy and myopathy, worsening of prior psychiatric conditions, adrenal insufficiency and avascular necrosis of the femoral head or humeral head. The latter effect results from glucocorticoid-induced down-regulation of ACTH release, and the ensuing disequilibrium of the hypothalamus-pituitary-adrenal axis that eventually induces atrophy of the adrenal cortex, and thenceforth to osteoporosis, growth inhibition and hypogonadism.

### Adverse Skin Effects of Topical Corticosteroids

Corticosteroids aid in palliating psoriatic lesions; however, skin side effects are frequent and diverse. The most prevalent are thinning of the epidermis and dermis (skin atrophy), the appearance of stretch marks, and altered cicatrization, while erythema, perioral dermatitis, hypertrichosis, acne and telangiectasis can also develop. Furthermore, steroids inhibit epidermal lipid synthesis eventually leading to an impaired epidermal barrier, adding to the barrier damage already inflicted by psoriasis. This in turn augments trans-epidermal water loss and reduces hydration aggravating skin dryness and irritation.

In addition, the face, axillae, groin and the area under the breasts are especially sensitive to the adverse effects of topical corticosteroids, including those of lower potency, which may promote facial telangiectasia and the formation of stretch marks (striae) at the rest of the above mentioned susceptible sites.

Of note, in comparison with adult psoriasis patients, susceptibility to the induction of topical systemic side effects by topical corticosteroids is higher in children, including the suppression of the hypothalamic-pituitary adrenal axis. This is due to the larger BSA-to-weight ratio displayed by children and infants, who—in consequence—are commonly treated with lower-potency corticosteroids.

Additional drawbacks of topical corticosteroid therapy are a) the fast decline of the response to prolonged topical application, that reduces the constricting capacity of dermal capillaries, and requires more frequent corticosteroid application or higher doses to attain an adequate effect; and b) aggravation of psoriasis when a patient abruptly abandons the treatment.

Use of Topical Corticosteroids Combined with Other Topical Agents.

One of the approaches to counteract corticosteroid side effects consists of using combined treatments where two therapeutic compounds target different cellular functions (epidermal differentiation and proliferation, immune cell functions, inflammation) to reduce skin lesions, while diminishing side effects.

The compounds most frequently combined with topical corticosteroids are vitamin D analogs, salicylic acid and retinoids, all of which have different mechanisms of action.

By acting on keratinocyte- and lymphocyte-vitamin D receptors, vitamin D analogues mitigate epidermal hyperproliferation, keratinization and neoangiogenesis, promote inflammatory cell apoptosis, reduce IL-1 and IL-6 levels, and restrict epithelial cell proliferation. Vitamin D analogues also induce the expression of antimicrobial peptides. Many of these actions neutralize the skin atrophy induced by corticosteroids.

Topical salicylic acid has keratolytic properties and its mechanism of action seems to involve breaking the bonds between adjacent keratinocytes and weakening the stratum corneum, i.e. the outermost layer of the epidermis. Salicylic acid used in combination with mild-corticosteroids improve skin penetration.

After binding the retinoic acid receptor (RAR), retinoids regulate gene transcription resulting in the decrease of keratinocyte proliferation, normalization of keratinocyte differentiation, and reduced inflammation.

In psoriasis, the preferred combination of topical agents is that consisting of vitamin D analogues and corticosteroids, which exhibits higher efficacy versus either monotherapy. However, in approximately 35% of the patients erythema, skin dryness, irritation, peeling and edema may occur. The vitamin D analogue most amply prescribed for combination treatment with corticosteroids is calcipotriol; however, only some corticosteroids can be combined with calcipotriol since the latter is inactivated when in contact with several of the corticosteroids.

The combination of topical corticosteroids and the retinoid tazarotene is also a successful psoriasis treatment with better efficacy than tazarotene alone. In this combined therapy, corticosteroids improve the efficacy and minimize tazarotene toxicity, whereas tazarotene diminishes corticosteroid-related skin atrophy.

Finally, psoriasis therapy frequently includes the use of corticosteroids combined with UVB irradiation, traditional systemic compounds (the retinoid acitretin, the immunosuppressant cyclosporine, and the immunosuppressant/anti-inflammatory methotrexate), and biological agents.

In conclusion, the above as a whole point to the adverse effects of corticosteroids as a hurdle to the treatment of psoriasis. Eliminating these unwanted effects while preserving corticosteroid efficacy remains a difficult endeavor for investigators. In this context, the present invention proposes the combination of corticosteroids and brassinosteroid analogs as a means of attaining anti-psoriasis efficacy by complementing the actions of both compounds, while reducing the unwanted effects associated to corticosteroid therapy by reducing its dosage or the time of corticosteroid treatment.

### Supporting Evidence

### 1. The Compounds of the Invention Activate LXR-Alpha Receptors

Since we had previously shown that the beneficial effects of the compounds of the invention are independent of glucocorticoid receptor (GR) activation, we set forth to investigate if they might exert their actions through activation of liver X receptors (LXR). For this purpose we used cultured cells transiently transfected with LXR-alpha, the retinoid X receptor (RXR) and a LXR response element-luciferase reporter (LRE-LUC).

### Methods:

A. Human embryonic kidney cells (HEK293T; 5×10⁵ cells/well) were cultured in DMEM medium supplemented with 10% (v/v) fetal bovine serum, 100 ug/ml of streptomycin, 100 IU/ml of penicillin and 2 mM glutamine. For the transient transfections, the cell cultures were added with plasmids carrying LXR-alpha (pLXR-alpha; 0.6 ug), the retinoid X receptor (pRXR; 0.2 ug), a LXR response element-luciferase reporter (pRE-LUC; 0.7 ug) and beta-galactosidase gene (pRSV-LacZ; 0.6 ug) as a transfection control, using Lipofectamine 2000 (Invitrogen). After transfection, the culture medium was replaced by serum-free DMEM, and the cells were incubated for 18 h with any of Compounds I through VI at 10⁵ M each, or vehicle [DMSO, 0.1% (v/v) final concentration], or the commercial LXR-alpha/LXR-beta agonist GW3965 at a concentration of 10⁻⁶ M. Compounds III to VI are stigmastane analogs bearing a fluorine atom at C-6. To assess LXR-alpha activation, at the end of the incubation period luciferase activity was measured by use of a luciferase assay system (Promega). The results of three independent assays, carried out in duplicate for each condition, are reported.

B. Baby hamster kidney cells (5×10⁵ cells/well) were cultured and transiently transfected as described above (Methods A.) for HEK293T cells. After transfection, the culture medium was replaced by serum-free DMEM, and the cells were incubated for 18 h with either Compound I or Compound II at 1×10⁻⁶ M, 3×10⁻⁶ M or 10⁻⁵ M each, or vehicle [DMSO, 0.1% (v/v) final concentration], or GW3965 at a concentration of 10⁻⁶ M. LXR-alpha activation was determined as described in A.

### Results:

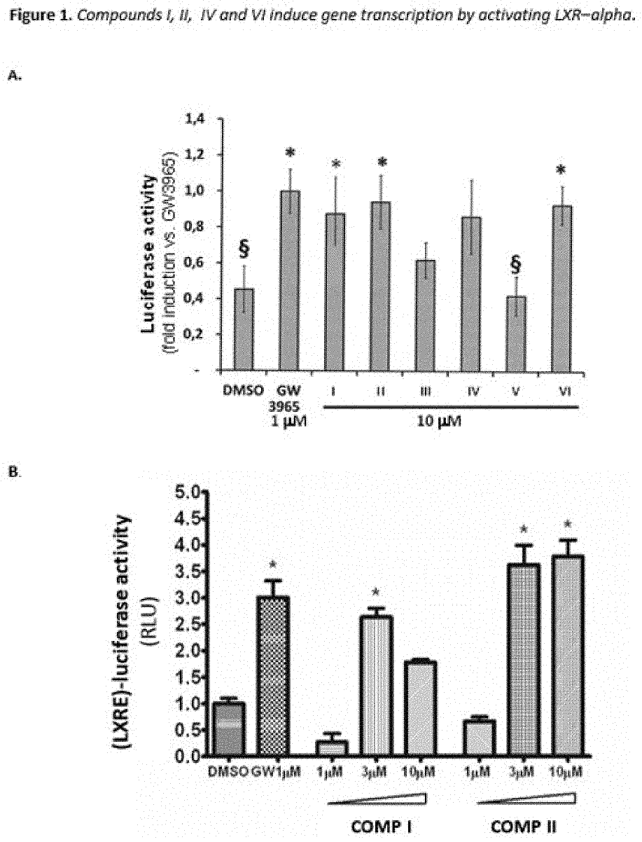

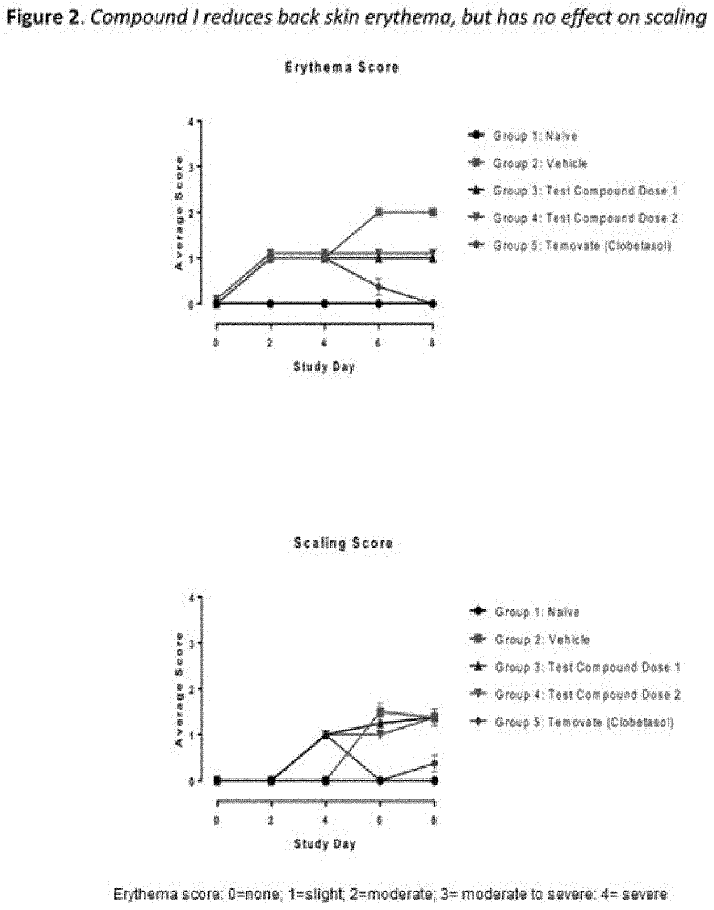

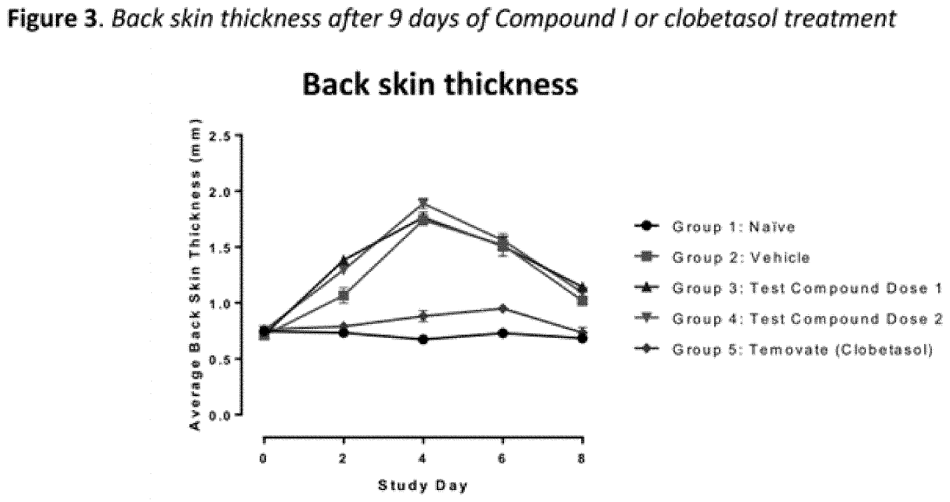

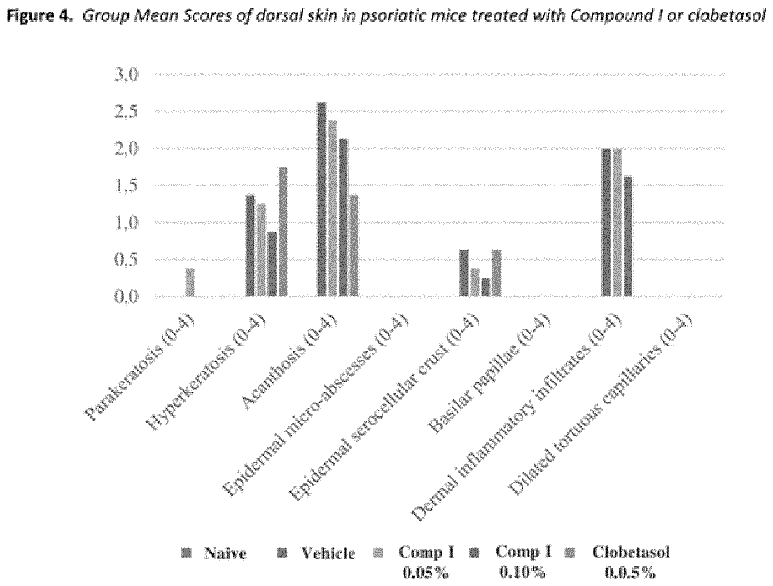

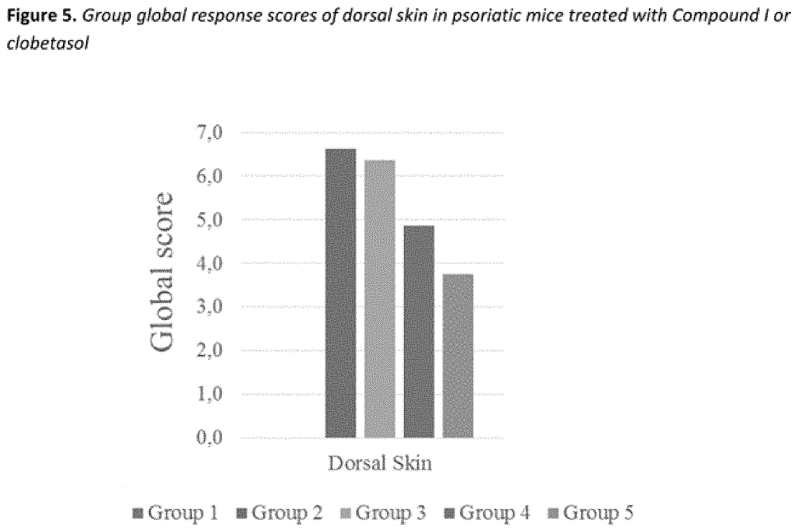

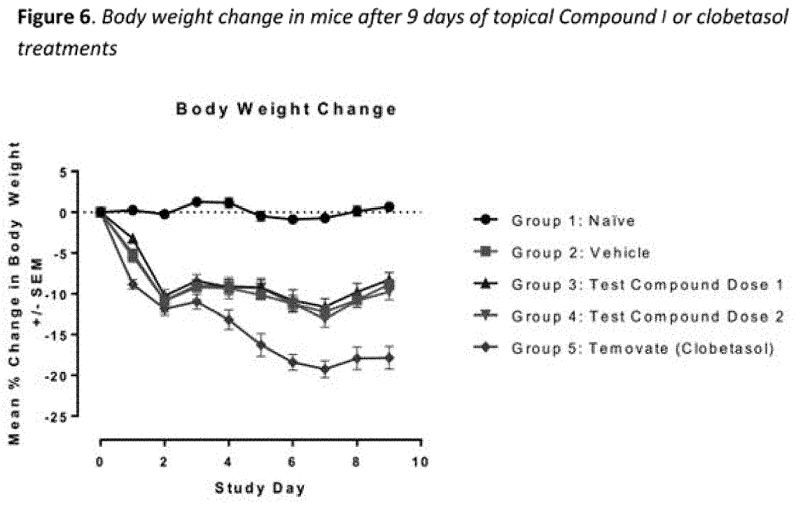

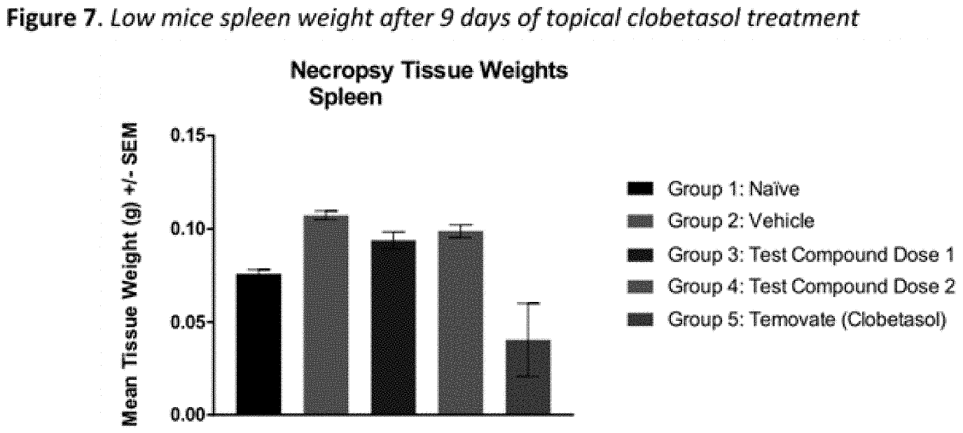

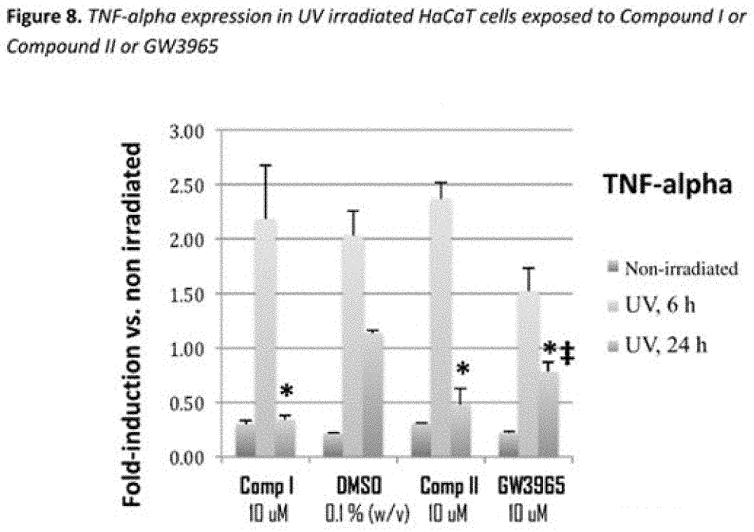

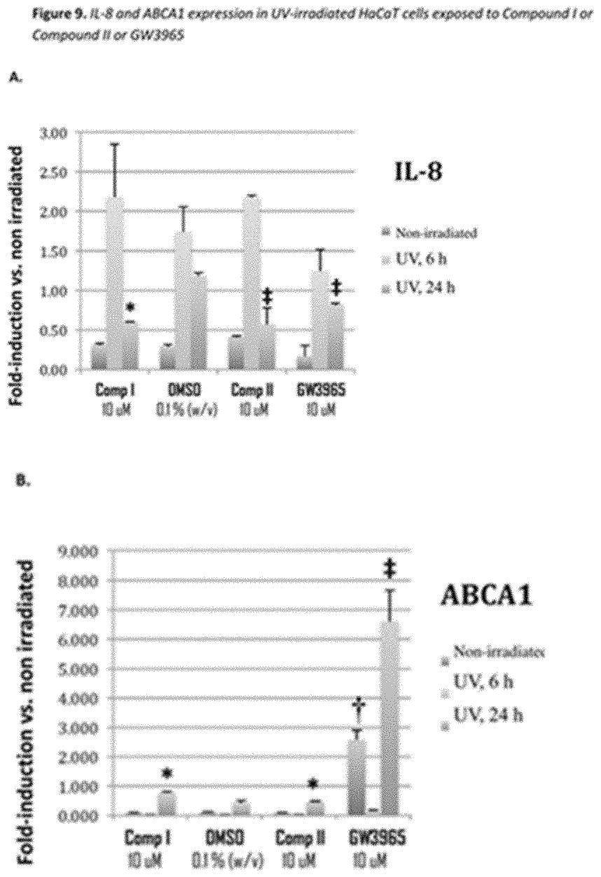

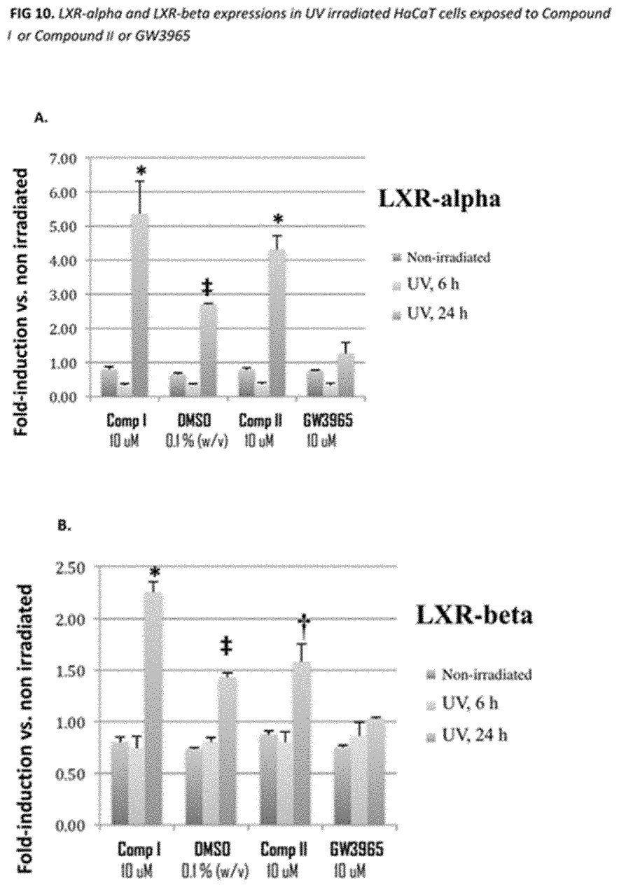

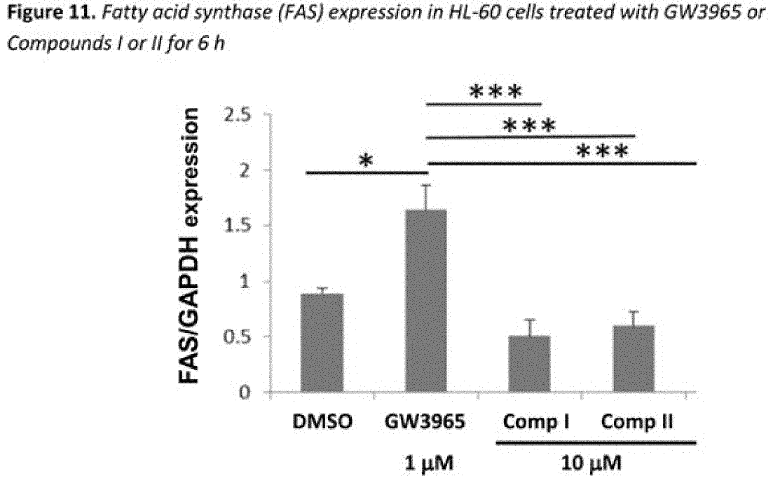

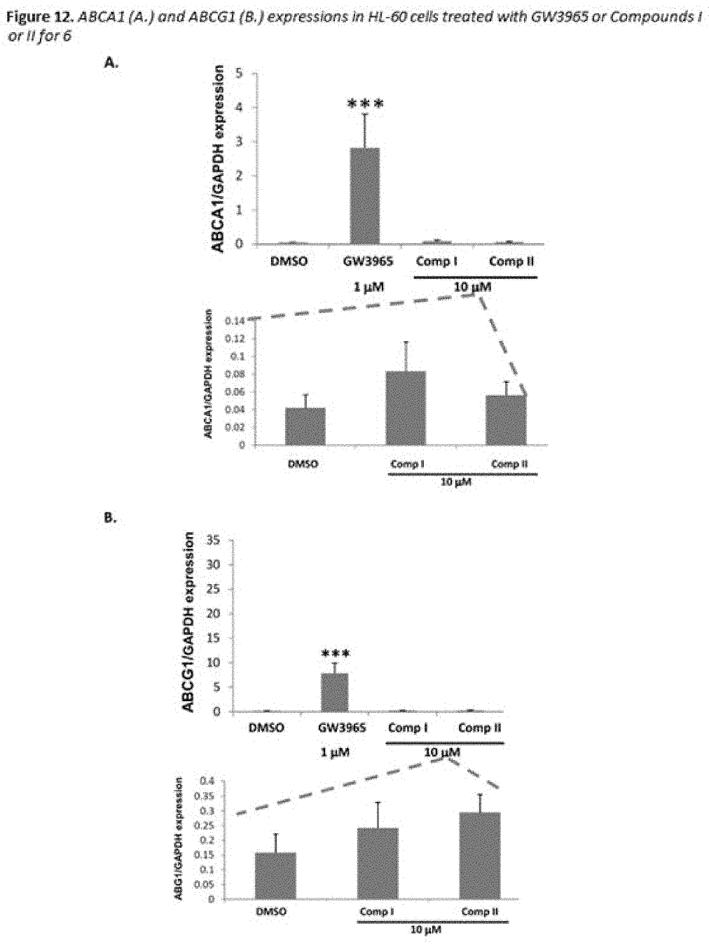

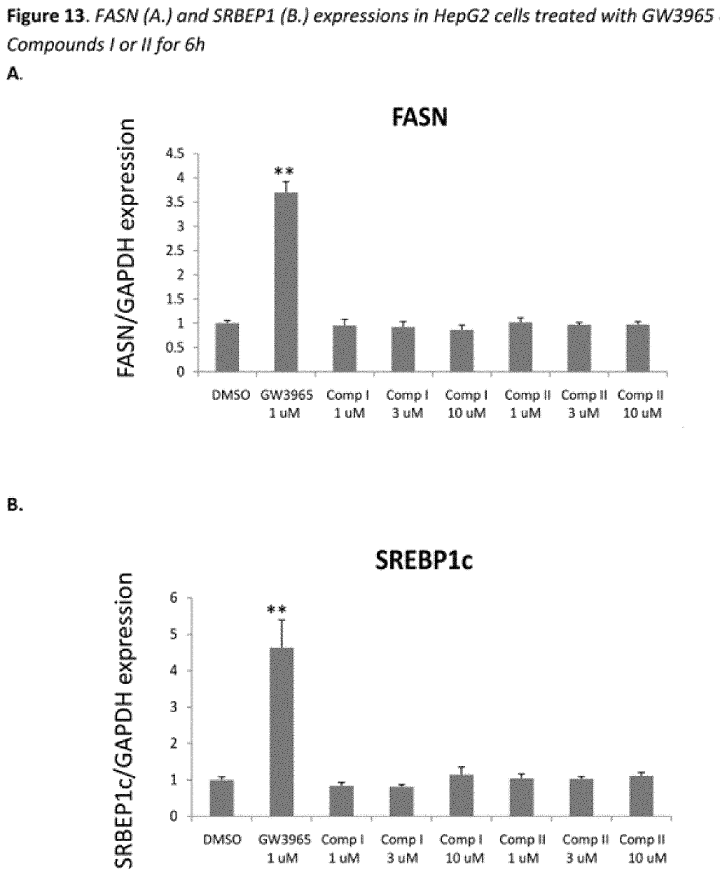

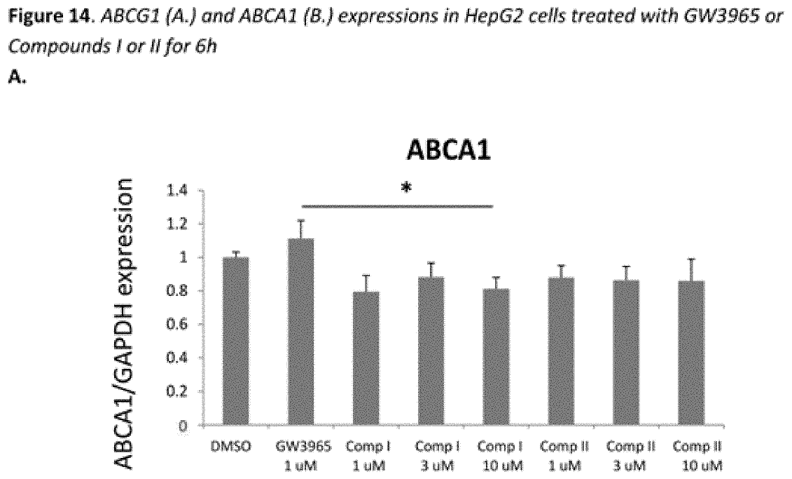

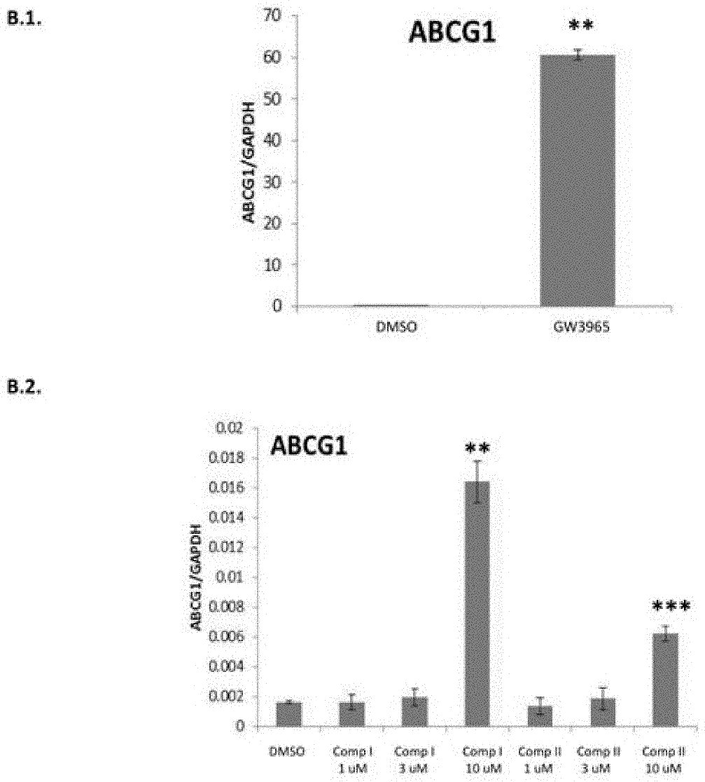

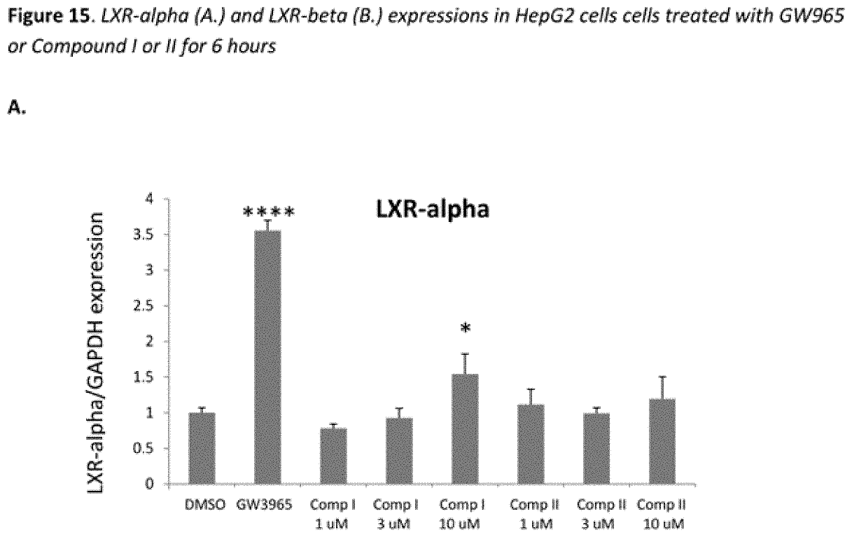

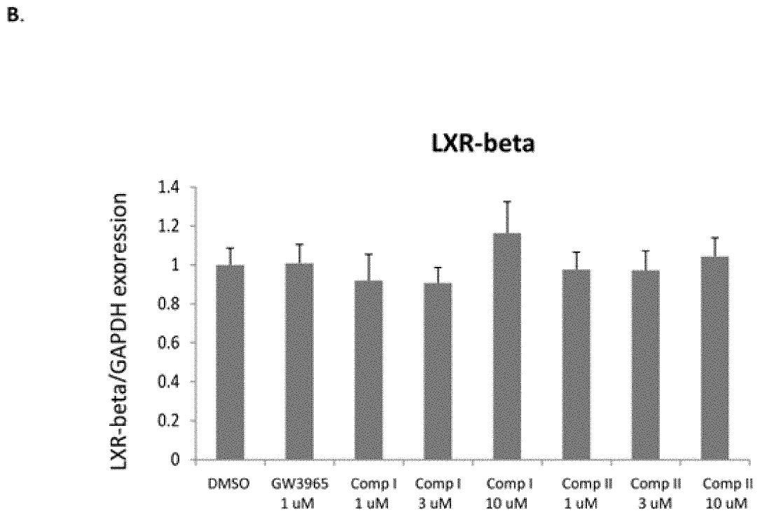

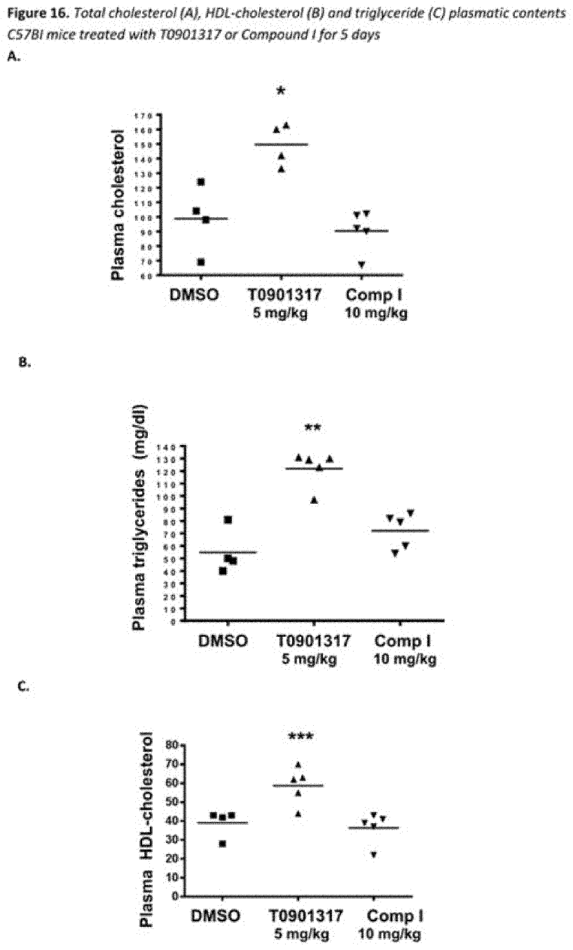

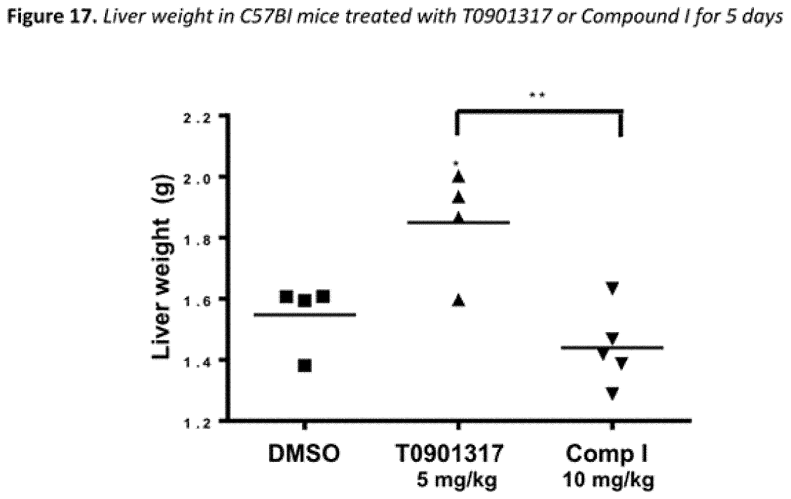

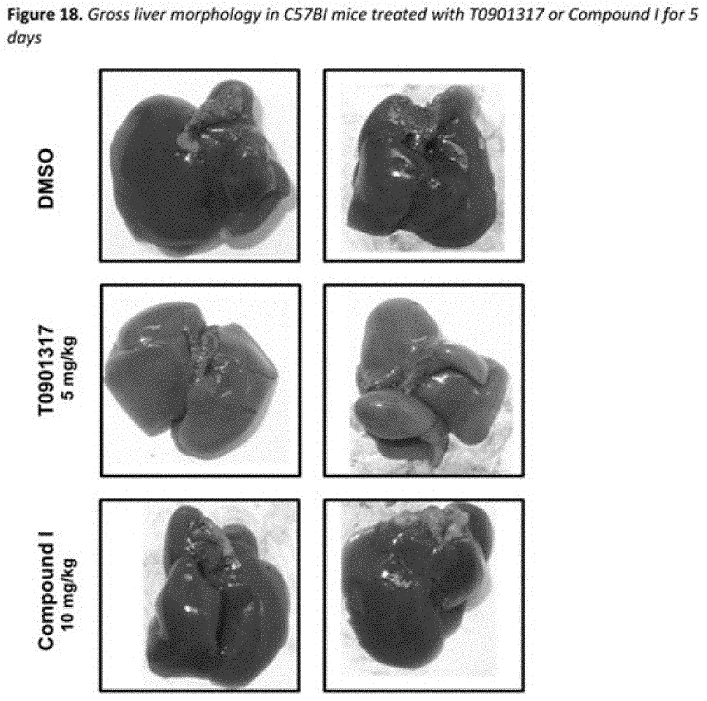

GW3965 (1 uM) induced a 3-fold increase in LXR-alpha driven gene expression compared with vehicle-treated cells; the induction of gene expression by 10 uM of either Compounds I, II, IV or VI was within the same range as that observed for GW3965 (FIG. 1, A). Compound I at a concentration of 3 uM, and Compound II at 3 uM and 10 uM induced a 3-fold increase LXR-alpha-driven luciferase expression compared with vehicle-treated cells, reaching the same level of induction as 1 uM GS3965. In contrast, Compound I or Compound II at concentrations of 1 uM had no effect on the LXR-alpha-driven expression of luciferase (FIG. 1, B).

### 2. The Compounds of the Invention Reduce the Signs of Psoriasis

To test whether Compound I might ameliorate the signs of psoriasis, we topically administered Compound I to imiquimod-induced skin lesions in mice. The imiquimod mouse model of psoriasis-like skin inflammation is characterized by lesions that show augmented epidermal cell proliferation, abnormal keratinocyte differentiation, neutrophil accumulation in epidermal microabcesses, and neoangiogenesis.

For this study, thirty two 8-week old female Balbc/J mice were housed individually in positively ventilated polysulfonate cages with HEPA filtered air at a density of 4 mice per cage. The animal room was lighted entirely with artificial fluorescent lighting, with a controlled 12 h light/dark cycle (6 am to 6 pm light). The normal temperature and relative humidity ranges in the animal rooms were 22±4° C. and 50±15%, respectively. The animal rooms were set to have a minimum of 15 air exchanges per hour. Filtered tap water, acidified to a pH of 2.5 to 3.0, and standard rodent chow were provided ad libitum. After 1 week of acclimation mice were randomized into 4 groups (n=8/each) as shown below (Table 1).

TABLE 1
Experimental scheme of mouse treatments in the imiquimod-induced psoriasis model

| | # Mice per | | | Dose conc./ | Dosing |
|---|---|---|---|---|---|
| Group | study arm | Compound to be used | Dosing Route* | volume/mouse | Frequency |
| 1 | 8 | Vehicle | Topical Cream | tbd | QD~9 days |
| 2 | 8 | Test Compound Dose 1 | Topical | tbd | QD~9 days |
| 3 | 8 | Test Compound Dose 2 | Topical | tbd | QD~9 days |
| 4 | 8 | Temovate (Clobetasol) | Topical | ~0.05 mg/mouse | QD~9 days |
| | | | 0.05% Cream | | |

Mice underwent procedures as detailed below:

1. IMQ induction: On day 0 all grouped mice received a topical dose of 62.5 mg of IMQ cream (5%) on the shaved back for 6 consecutive days. (Day 0 to Day 5)

2. Mice were dosed with 0.1-0.2 ml of vehicle [90% (v/v) DMSO in pyrogen-free sterile water] or test compounds (0.1-0.2 ml of Compound I) or reference compound (˜0.05 mg clobetasol propionate, 0.5 mg/g cream) for 9 days (day 0-day 8) as indicated in Table 1. Compound I Doses 1 and 2 were 0.05% (w/v) and 0.1% (w/v) in 90% (v/v) DMSO in vehicle.

3. Mice were scored for clinical parameters of disease based on degree of erythema, scaling and thickening independently on a scale of 0-4. 0=none; 1=slight; 2=moderate; 3=moderate to severe: 4=severe. These observations were recorded on day 0, 2, 4, 6, and 8. On the same days, back skin thickness was measured with calipers.

4. Body weights were measured daily.

5. Mice were sacrificed on day 10 by CO₂ asphyxiation.

6. A part of the back skin was harvested and fixed for histology. Slides were processed, stained with H&E for histology assessment. The histopathology scoring system is shown in the following Table 2.

7. Spleens were harvested, weighed and discarded.

### Results:

For mice exposed to IMQ, 9 days after initiation of treatments skin erythema was moderate in all untreated controls whereas scaling was moderate in ⅜ and slight in ⅝ animals. On day 8, both assayed Compound I doses were associated with slight erythema in all the animals, representing a 50% reduction relative to vehicle-treated mice and pointing to the amelioration of this sign, whereas scaling showed no improvement when compared to vehicle-treated controls (FIG. 2). On day 8, erythema was absent in all clobetasol-treated mice and scaling was absent in ⅝ mice and slight in the rest of the animals (FIG. 2).

None of the assayed Compound I's doses prevented back skin thickening, whereas clobetasol treatment inhibited IMQ-induced skin thickening in the back (FIG. 3).

Dorsal skin sections were evaluated histopathologically for evidence of key features of psoriasis, such as parakeratosis, hyperkeratosis (thickening of the stratum corneum), acanthosis (diffuse thickening of the stratum spinosum of the skin), epidermal serocellular crusts, epidermal microabscesses, basilar papillae, dermal inflammatory infiltrates and dilated tortuous capillaries (Table 2. Histopathological scoring system and definition used in the Imiquimod psoriasis model).

Group mean response scores for the microscopic findings in dorsal skin are shown in Table 3, and representative graphs in FIGS. 4 and 5.

Summarizing, the dorsal skin from naive animals were all within histological normal limits.

Experimental psoriasis animals that were treated with either vehicle or Compound I, 0.05% (w/v) or Compound I, 0.10% (w/v) or Clobetasol 0.05% (w/w) displayed varying degrees of dorsal skin pathology, as indicated by evaluation of inflammatory and proliferative epidermal and dermal psoriasis changes (acanthosis, hyperkeratosis) and dermal inflammatory infiltrates, which were generally the most common microscopic alterations. Samples from vehicle-treated psoriatic controls were generally the most severely affected. Samples from 0.05% (w/v) Compound I-treated mice generally had slightly lower severity of microscopic alterations, relative to vehicle-treated psoriatic controls. In samples from 0.10% (w/v) Compound I-treated mice the severity of microscopic alterations was lower than in samples from vehicle-treated psoriatic controls and 0.05% (w/v) Compound I-treated mice. The lowest severity of microscopic alterations was associated to clobetasol treatment. These observations are summarized in Table 3 (Group Mean Scores) and FIG. 4; the resulting global scores are shown in Table 5 and FIG. 5.

TABLE 2
Histopathological scoring system and definition, as
used in the imiquimod psoriasis model in mice

| Criterion for Each | | | | Epidermal |
|---|---|---|---|---|
| Histopathology | | | | serocellular |
| Score | Parakeratosis | Hyperkeratosis | Acanthosis | crusts |
| Grade0 | There are no | There is no | There are no | There are no |
| NONE | visible foci of | discernible | visible foci of | visible foci of |
| | keratin layers | increase in the | hyperplasia of | degenerate |
| | with retention of | thickness of the | the squamous | leukocyte |
| | keratinocyte | keratin layer. | cell layer of the | accumulation |
| | nuclei over the | | epidermis. | within the |
| | epidermal | | | squamous cell |
| | surface. | | | layer of the |
| | | | | epidermis. |
| Grade1 | There are rare | There is less | There are rare | There are rare |
| MINIMAL | foci. | than 15% | foci with slight | foci. |
| | | increase in the | to prominent | |
| | | thickness of the | thickening of | |
| | | keratin layer. | the epidermis. | |
| Grade2 | There are a | There is 15 to | There are a few | There are a |
| MILD | few foci. | 30% increase in | foci with | few foci. |
| | | the thickness of | prominent | |
| | | the keratin layer. | thickening of | |
| | | | the epidermis. | |
| Grade3 | There are | There is 30 to | There are | There are |
| MODERATE | multiple foci. | 60% increase in | multiple foci | multiple foci. |
| | | the thickness of | with prominent | |
| | | the keratin layer. | thickening of | |
| | | | the epidermis. | |
| Grade4 | There are | There is greater | There are | There are |
| SEVERE | extensive foci | than 60% | extensive foci | numerous foci |
| | invoking more | increase in the | involving more | invoking more |
| | than 70% of the | thickness of the | than 70% of the | than 70% of |
| | epidermal | keratin layer | epidermal | the epidermal |
| | surface. | invoking more | surface with | surface. |
| | | than 70% of the | marked | |
| | | epidermal | thickening of | |
| | | surface. | the epidermis. | |

| Criterion for Each | Epidermal | | Dermal | Dilated |
|---|---|---|---|---|
| Histopathology | micro- | Basilar | inflammatory | tortuous |
| Score | abscesses | papillae | infiltrates | capillaries |
| Grade0 | There are no | There are no | There are no | There are no |
| NONE | visible foci of | visible foci of | visible foci of | visible foci of |
| | discrete | rete-ridge like | inflammatory | dilated tortuous |
| | aggregates of | papillary | cell | capillaries within |
| | neutrophils | extensions of | aggregates in | the dermis. |
| | and | the | the dermis. | |
| | lymphocytes | hyperplastic | | |
| | within the | epidermis into | | |
| | squamous cell | the dermal | | |
| | layer of the | stroma. | | |
| | epidermis. | | | |
| Grade1 | There are rare | There are rare | There are rare | There are rare |
| MINIMAL | foci. | foci. | foci. | foci. |
| Grade2 | There are a | There are a | There are a | There are a |
| MILD | few foci. | few foci. | few foci. | few foci. |
| Grade3 | There are | There are | There are | There are |
| MODERATE | multiple foci. | multiple foci. | multiple foci. | multiple foci. |
| Grade4 | There are | There are | There are | There are |
| SEVERE | numerous foci | numerous foci | extensive foci | numerous foci |
| | involving more | invoking more | involving more | involving more |
| | than 70% of | than 70% of | than 70% of | than 70% of |
| | the epidermal | the dermis. | the dermis. | the dermis. |
| | surface. | | | |

The following Table 3 shows that among the four back skin parameters that were altered in the present psoriasis model (hyperkeratosis, acanthosis, epidermal sero-cellular crust, and dermal inflammatory infiltrates), hyperkeratosis and epidermal sero-cellular crust were reduced by 50% in 0.1% (w/v) Compound I relative to 0.1% (w/v) Clobetasol-treated mice, whereas acanthosis was reduced by 33% and dermal inflammatory infiltrates were absent in Clobetasol-treated versus 0.1% (w/v) Compound I-treated animals. Epidermal serocellular crusts appear when plasma exudes through an eroded epidermis, and a consolidated mass of cellular debris, dried exudate and serum forms an outer layer. As already mentioned, hyperkeratosis refers to the thickening of the stratum corneum, i.e., the outermost layer of the epidermis. The stratum corneum consists of non-living cells called corneocytes, that originate from the transformation of keratinocytes. The stratum corneum forms a barrier that protects the underlying tissue from dehydration, infection, chemicals and mechanical stress. Therefore, Compound I and Clobetasol treatments provide complementary skin protection, pointing to an improved response for administration of a Compound I-Clobetasol combination treatment.

TABLE 3
Group mean scores of dorsal skin in psoriatic mice
treated with Compound I or clobetasol

| | | | Comp I | Comp I | Clobetas, |
|---|---|---|---|---|---|
| Tissue Response | Naive | Vehicle | 0.05% | 0.10% | 0.0.5% |
| Parakeratosis (0-4) | 0.0 | 0.0 | 0.4 | 0.0 | 0.0 |
| Hyperkeratosis (0-4) | 0.0 | 1.4 | 1.3* | 0.9** | 1.8 |
| Acanthosis (0-4) | 0.0 | 2.6 | 2.4 | 2.1 | 1.4†† |
| Epidermal serocellular | 0.0 | 0.6 | 0.4 | 0.3† | 0.6 |
| rust (0-4) | | | | | |
| Epidermal micro- | 0.0 | 0.0 | 0.0 | 0.0 | 0.0 |
| abscesses (0-4) | | | | | |
| Basliar papillae (0-4) | 0.0 | 0.0 | 0.0 | 0.0 | 0.0 |
| Dermal inflammatory | 0.0 | 2.0 | 2.0 | 1.5 | 0.0†† |
| infiltrates (0-4) | | | | | |
| Dilated tortuous | 0.0 | 0.0 | 0.0 | 0.0 | 0.0 |
| capillaries (0-4) | | | | | |

*p = 0.0455 vs. Clobetas. 0.05%;
**p = 0.0075 vs. Clobetas. 0.05%;
††p = 0.0075 vs. Comp I 0.05% and 0.10%;
†p = 0.008 vs. Clobetas. 0.05% (Chi Square)

The complementarity of Compound I and clobetasol treatments is displayed in the following Table 4 that presents a summary of the results shown in the above Table 3, and where the (+) sign represents the presence of a protective effect, while a (−) sign represents the absence of protective effect:

TABLE 4

| | Comp I | Clobetasol |
|---|---|---|
| Tissue Response | 0.10% | 0.05% |
| Hyperkeratosis | + | − |
| Epidermal | + | − |
| serocellular crust | | |
| Acanthosis | − | + |
| Dermal inflammatory | − | + |
| infiltrates | | |

FIG. 4 shows Group Mean Scores of dorsal skin in psoriatic mice treated with Compound I or clobetasol. Group global response scores are shown in Table 5.

TABLE 5
Group global response scores of dorsal skin in
psoriatic mice treated with Compound I or clobetasol

| Mean Global | | | Comp I | Comp I | Clobetas. |
|---|---|---|---|---|---|
| Score | Naive | Vehicle | 0.05% | 0.10% | 0.05% |
| Dorsal skin | 0.0 | 6.6 | 6.4 | 4.9 | 3.8 |

FIG. 5 is a graphic representation of the Group global response scores of dorsal skin shown in Table 5.

2a. Observed Adverse Effects of Corticosteroid Treatment

However, the adverse effects of topical corticosteroid treatment were evident in mice topically treated with clobetasol for 9-days that showed a 15% reduction of body weight(FIG. 6) and a 62.6% reduction of spleen weight (FIG. 7), compared to the vehicle-treated psoriatic mice. The reductions of body and spleen weights represent two well-known secondary effects of glucocorticoids in rodents, which seem to be related to inhibition of food intake and to the catabolic properties of this type of compounds. In Compound I-treated animals body weight was 8% lower than in naïve mice, and there was no change relative to vehicle-treated animals, whereas in Compound I-treated -treated animals spleen weight was 20% higher than in naïve mice, and showed no change relative to vehicle-treated animals.

Currently, topical corticosteroids are the backbone of anti-psoriasis pharmacologic arsenal (see above Background of the Invention) but their use is limited by the accompanying adverse effects, including skin atrophy, telangiectases and/or striae, and secondary systemic effects (see above Background of the Invention). To counteract these unwanted consequences, discontinuous or pulse corticosteroid dosing, as well as reducing topical corticosteroid dosage as a result of combining with other topical compounds, are suggested to increase treatment efficacy and corticosteroid safety for longer use.

In conclusion, we provide here the rationale for the use of either Compound I to VI alone or in combination with glucocorticoids for topical and systemic treatment of psoriasis. The benefit of the combination therapy is indicated by the complementary action of Compound I and clobetasol in the treatment of psoriasis (see Supporting Evidence, Section 2, Tables 3 and 4), and the superior effect shown by a Compound I—clobetasol combination treatment in reducing the signs of psoriasis (see below Section 2b).

Compound I's lack of the adverse effects of LXR agonists underscores the benefits of this invention; particularly considering that the severe adverse effects of corticosteroids limit their continued therapeutic use. On the other hand, at present the drawbacks of the existing LXR agonists have impeded their successful therapeutic use.

2b. Superior Effect of a Compound I—Clobetasol Combination Treatment in Reducing the Signs of Psoriasis

To test whether a Compound I-clobetasol combination treatment might provide better protection against psoriatic skin lesions than clobetasol when applied alone, we used the same imiquimod-induced psoriasis model as described in SUPPORTING EVIDENCE-Section 2., entitled The compounds of the invention reduce the signs of psoriasis.

Mice were dosed with 0.1-0.2 ml of vehicle [90% (v/v) DMSO in pyrogen-free sterile water], or 0.1% (w/v) Compound I- 0.05% (w/v) clobetasol combination in 90% (v/v) DMSO, or reference compound (˜0.05 mg clobetasol propionate, 0.5 mg/g cream) for 9 days.

The reductions of erythema, scaling and back skin thickness were significantly more marked in Compound I-clobetasol combination-treated mice than in clobetasol-treated animals. Also, histopathological analysis of back skin samples showed that hyperkeratosis, acanthosis, epidermal serocellular crusts and dermal inflammatory infiltrates were improved in mice treated with the Compound I-clobetasol combination versus those treated with clobetasol alone.

This evidence indicates the superiority of a Compound I—clobetasol combination as a therapeutic agent in psoriasis.

3. The Compounds of the Invention Provide Protection in Photoaging: In UV-Irradiated HaCaT Cells, Compounds I and II, Reduce the Expression of Proinflammatory Cytokines (TNF-Alpha and IL-8), and Stimulate the Expression of LXR-Alpha and LXR-Beta; in Addition, they Marginally Induce ABCA1

Solar UV damages the skin by chronically producing low level inflammation mediated by the release of pro-inflammatory cytokines (TNF-alpha and IL-8), MMPs and cyclooxygenase-2 (COX-2) [see above, Background of the Invention, “UV, photoaging and LXR”]. It is known that the synthetic LXR agonist T0901317 inhibits UV-induced skin damage and wrinkle formation in a mouse model of photoaging; however, T0901317 also induces hypertriglyceridemia and hepatic steatosis. The present invention establishes that Compounds I and II can protect keratinocytes from UV-induced damage (i.e., reduce the inflammatory response) without the adverse effects of LXR agonists (see Section 4).

### Methods:

HaCaT cells—a human immortalized keratinocyte cell line—were exposed to 15 J/m² UVB (254 nm) irradiation. Twenty four hours after irradiation, the cells were harvested and RNA isolated to assess the expression of LXR-alpha, LXR-beta, ABCA1, TNF-alpha, IL-6, and IL-8 genes. HaCaT cells were cultured in DMEM supplemented with 10% (v/v) fetal bovine serum, penicillin (100 U/mL), streptomycin (100 mg/mL) and glutamine (2 mM).

After preincubating the cells for 1 h in the presence of either Compound I (10 uM) or Compound II (10 uM), or GW3965 (10 uM), the culture medium was aspirated and the cells were exposed to UV radiation (254 nm; 15 J/m²); immediately after, the culture media were replenished, and after 6 h or 24 h, the cells were collected for RNA isolation and purification, followed by retro-transcription and real time PCR amplification of cDNA, as described in Section 5a. entitled “Compounds I and II fail to induce FAS, ABCA1 and ABG1 in HL-60 cells”. The expressions of ABCA1, TNF-alpha, IL-6, IL-8, LXR-alpha, and LXR-beta were normalized according to glyceraldehyde-3-phosphate dehydrogenase (GAPDH) expression. The results of three independent assays, carried out in duplicate for each condition, are reported.

### Results:

Twenty-four hours after UV irradiation, TNF-alpha expression was significantly lower in cells incubated with Compounds I or II or with GW3965 versus DMSO-treated cells [*p=0.032 vs. DMSO (UV, 24 h), Mann Whitney; FIG. 8]. Also, in GW3965-treated cells TNF-alpha expression was significantly higher than in Comp I-treated cells [‡p=0.045 vs. Comp I (UV, 24 h), Mann Whitney; FIG. 8. Twenty four hours after UV irradiation, IL-8 expression was significantly lower in cells incubated with Compounds I or II [*p=0.034 vs. DMSO (UV, 24 h), Mann Whitney; FIG. 9, A] or GW3965 versus DMSO-treated cells [‡p=0.047 vs. DMSO (UV, 24 h), Mann Whitney; FIG. 9, A]. In all groups, ABCA1 expression at 24 h after UV irradiation was higher than in DMSO-treated cells (*p=0.038, Mann Whitney; FIG. 9, B); however, the induction of ABCA1 expression in GW3965-treated cells was stronger than in Comp I- or Comp II-treated cells, in both non-irradiated HaCaT (†p=0.024, Mann Whitney; FIG. 9, B) and 24 h after UV exposure (‡p=0.001, Mann Whitney; FIG. 9, B).

Twenty four hours after UV-irradiation, LXR-alpha expression was higher in HaCaT incubated in the presence of DMSO compared with non-irradiated cells and GW3965-treated cells (‡p=0.036, Mann Whitney; FIG. 10, A). However, both Compound I and Compound II stimulated LXR-alpha expression even further [*p=0.021 vs. DMSO (UV, 24 h) and GW3965 (UV, 24 h), Mann Whitney; FIG. 10, A]. LXR-beta expression responded to UV irradiation and to incubation with either Compound I, or Compound II, or GW3965, or DMSO in the same direction as LXR-alpha expression, although the magnitude of the changes were less marked [‡p=0.044 vs. non-irradiated (UV, 24 h) and GW3965-treated Ha-Cat (UV, 24 h), Mann Whitney; FIG. 10, B]. Compound I stimulated LXR-beta expression even further [*p=0.032 vs. DMSO (UV, 24 h) and GW3965 (UV, 24 h) Mann Whitney; FIG. 10, B]. In Compound II-treated cells, LXR-beta induction at 24 h after UV irradiation was more modest than that observed for Compound I [†p=0.040 vs. GW3965 treated cells (UV, 24 h)]

Summarizing, Compounds I and II marginally induced ABCA1 in UV-irradiated HaCaTcells, but they reduced the expression of TNF-alpha and IL-8, and stimulated the expression of LXR-alpha and LXR-beta.

Considering the results shown in FIGS. 8 and 9, the compounds of the invention are suitable to be used for protection from the effects of photoaging.

### 4. The Compounds of the Invention do not Display LXR Agonist UNWANTED SECONDARY EFFECTS

4a. Compounds I and II Fail to Induce FAS, ABCA1 and ABCG1 in HL-60 Cells

To investigate the effects of Compounds I and II on the expression of three genes involved in lipid metabolism [fatty acid synthase (FAS); and two ATP-dependent cholesterol and phospholipid transporters, such as ABCA1 and ABCG1], human promyelocytic leukemia cells (HL-60) that endogenously express LXR-alpha and LXR-beta were incubated in the presence of either Comp I or Comp II (10 uM), or GW3965 (1 uM) for 6 h. mRNA levels for FAS, ABCA1 and ABCG1 were determined by real time PCR and results were normalized to GAPDH expression.

### Methods:

HL-60 (10⁶ cells/well) were cultured in RPMI medium supplemented with 5% (v/v) fetal bovine serum, penicillin (100 IU/mL), streptomycin (100 ug/mL) y cyprofloxacin (0.4 ug/mL). The cells were incubated for 6 h in the presence of either Compound I (10 uM), or Compound II (10 uM), or the commercial LXR agonist GW3965 (1 uM), or Vehicle [DMSO, 0.1% (v/v)]). To obtain RNA, at the end of the incubation the cells were collected by centrifugation at 1,000 rpm for 2 min, washed with phosphate buffered saline, and lysed in 300 ul TRIZOL (Invitrogen). RNA samples were suspended in ul of RNAse free water, and their concentration determined (NanoDrop spectrophotometer). The ratio of the absorbance at 260 and 280 nm was used to assess RNA purity of an RNA preparation, with a value of 1.8-2.0 indicating pure RNA. To obtain cDNA, 1 ug of total RNA was denatured by incubating for 5 min at 70° C., before the addition of a mixture containing 0.5 ug oligo-dT primers and 25 ng/ml of random primers (Invitrogen), 1.0 mM dNTPs (Invitrogen), and 200 U M-MLV reverse transcriptase (Promega); after this the mixture was retrotranscribed at 37° C. for 60 min, followed by an incubation at 95° C. for 5 min to inactivate the enzyme. For the real time PCR amplification (Stratagene Thermal Cycler), cDNA samples were diluted 1/5 to 1/10 depending on the target amplification product. Calibration curves were obtained by use of eight successive 1:2 sample dilutions. Amplifications were carried out in a 25 ul final volume (5 ul cDNA sample plus 20 ul of reaction mixture) in the presence of either 3, 4 or 5 mM MgCl₂ (depending on the amplified sequence), 0.25 mM dNTPs (Invitrogen), 1.25 U Taq polymerase (Invitrogen), 1 μM of the specific primers according to the target amplification sequence, and 0.025 μl SYBR Green (Roche). Melting curves were used to assess the specificity of the amplification products. All qPCR programs included 2 min denaturation at 95° C., followed by cycle repetitions with annealing temperatures as indicated in Table 6, and a final extension at 72° C. for 5 min.

### Results:

In GW3965-treated cells, FAS, ABCA1 and ABCG1 expressions were 1.8 times, ˜3 times, and ˜10 times higher than in vehicle-treated controls, respectively (p<0.05); in contrast, in Comp I- or Comp II-treated cells FAS mRNA was ˜40% lower (p<0.001) than in vehicle-treated cells, and none of the test compounds significantly affected ABCA1 and ABCG1 expressions (FIGS. 11 & 12).

TABLE 6
Primer sequences used for cDNA amplification

| | | Anneal- |
|---|---|---|
| | | ing |
| | | Temper- |
| | | ature |
| Gen | Primer sequence | (° C.) |
| ABCA1 | Fwd: | 60 |
| (ATP-binding | GAGTGAAGCCTGTCATCTACTG | |
| cassette, sub- | (SEQ ID NO: 1) | |
| family A, | Rev: | |
| member1) | GAGTGAAGCCTGTCATCTACTG | |
| | (SEQ ID NO: 2) | |
| ABCG1 | Fwd: | 64 |
| (ATP-binding | TCCTCTTCAAGAGGACCTTCCT | |
| cassette, sub- | (SEQ ID NO: 3) | |
| family G, | Rev: | |
| member1) | CCCAATGTGCGAGGTGAT | |
| | (SEQ ID NO: 4) | |
| FAS | Fwd: | 60 |
| (fatty acid | ACAGGGACAACCTTGGAGTTCT | |
| synthase) | (SEQ ID NO: 5) | |
| | Rev: | |
| | CTGTGGTCCCACTTGATGAGT | |
| | (SEQ ID NO: 6) | |
| TNF-alpha | Fwd: | 58 |
| (tumor necrosis | CTGCTGCACTTTGGAGTGAT | |
| factor alpha) | (SEQ ID NO: 7) | |
| | Rev: | |
| | ACGCTGCATAGCTCGTTC | |
| | (SEQ ID NO: 8) | |
| IL-8 | Fwd: | 62 |
| (interleukin-8) | CTGCGCCAACACAGAAATTA | |
| | (SEQ ID NO: 9) | |
| | Rev: | |
| | ATTGCATCTGGCAACCCTAC | |
| | (SEQ ID NO: 10) | |
| LXR-alpha | Fwd: | 64 |
| (Liver X | CCTTCAGAACCCACAGAGATCC | |
| receptor alpha) | (SEQ ID NO: 11) | |
| | Rev: | |
| | ACGCTGCATAGCTCGTTCC | |
| | (SEQ ID NO: 12) | |
| LXR-beta | Fwd: | 63 |
| (Liver X | TTTGAGGGTATTTGAGTAGCGG | |
| receptor beta) | (SEQ ID NO: 13) | |
| | Rev: | |
| | CTCTCGCGGAGTGAACTAC | |
| | (SEQ ID NO: 14) | |

4b. Compounds I and II Fail to Induce FAS, ABCA1 and SRBEP1 in HepG2 Cells

To investigate the effects of Compounds I and II on the expression of fatty acid synthase (FAS or FASN), serum response element binding protein-1 (SRBEP-1), ABCA1, ABCG1, LXR-alpha and LXR-beta in a human liver carcinoma cell line (HepG2) that endogenously express LXR-alpha and LXR-beta, the cells were incubated in the presence of either Comp I or Comp II (1 uM, 3 uM and 10 uM) or GW3965 (1 uM) for 6 h, with a methodology as described in section 4a.

### Results:

In GW3965-treated HepG2 FASN, SRBEP-1 and ABCG1 expressions were 3.7 times, 4.6 times and 60 times higher, respectively, than in vehicle (DMSO) treated controls (FIGS. 13 and 14). At the concentrations tested, Comp I and Comp II did not induce FASN, SRBEP-1 or ABCA1 expressions in these liver cells; however, Comp I and Comp II at 10 uM induced ABCG1 expression 8 times and 3 times, respectively (FIG. 14 B.2.). GW3965 (1 uM) and Comp I (10 uM) induced the expression of LXR-alpha 3.5 and 1.5 times, respectively, compared with DMSO treated HepG2 (FIG. 15 A). None of the compounds tested had an effect on LXR-beta expression.

In conclusion, the above evidence (FIGS. 13, 14 and 15) indicates that in HepG2 cells the compounds of the invention do not activate genes responsible for unwanted effects (FAS and SRBEP), while moderately inducing ABCG1 and LXR-alpha expressions. This was further confirmed a) in the assays shown in Section 3. where Compounds I and II marginally induced ABCA1 in UV-irradiated HaCaTcells, but they reduced the expression of TNF-alpha and IL-8, and stimulated the expression of LXR-alpha and LXR-beta, and b) in mice treated with Compound I for 5 days that showed no signs of hypertriglyceridemia or hepatic steatosis compared with T0901317-treated mice (see below Section 4c.)

4c. Compound I Fails to Induce Hepatic Steatosis in Mice

Administration of synthetic LXR agonists such as T0901317 induces a marked increment of liver lipogenesis that leads to hepatic steatosis and plasma hypertriglyceridemia. We investigated whether Compound I has adverse effects on lipogenesis when administered to mice at 10 mg/kg body weight, a dosage at which we previously showed that Compound I reduces granuloma formation in a mouse model of subchronic inflammation without the adverse effects of corticosteroids (not shown).

### Methods:

Male C57BI mice received daily for 5 days intraperitoneal injections containing either 10 mg Compound I/kg, or 5 mg T0901317/kg, or vehicle [90% (v/v) DMSO in apyrogenic sterile water]. On the sixth day of the assay, the animals were fasted for 4 h before decapitation. Blood plasma was obtained for the determination of total cholesterol, HDL-cholesterol and triglyceride contents. The livers were resected, and their weights and colors used as gross markers of increased liver triglyceride contents.

### Results:

In T0901317-treated mice total cholesterol, HDL-cholesterol and triglyceride plasmatic contents were significantly higher (↑50%, 50%, and >2-times, respectively) than in Compound I- or Vehicle-treated mice (FIG. 16). In addition, in T0901317-treated but not in Comp I-treated mice liver weight was 27% higher, and liver color paler than in DMSO-treated mice, pointing to T0901317 as a stimulator of liver triglyceride accumulation.

Based on the results shown in FIGS. 16, 17 and 18, as exemplified for Compound I at a dosage at which we previously showed that reduces granuloma formation in a mouse model of subchronic inflammation without the adverse effects of corticosteroids (not shown), the compounds of the invention when administered in vivo reduce both inflammation and the signs of psoriasis without the adverse effects of both corticosteroids and LXR agonists.

Available evidence shows that the potency of different LXR agonists is at least partly dependent on the corepressor/coactivator factor interactions they induce as a result of the specific conformational changes they promote upon binding LXRs. For example, the synthetic agonist LN6500 differs from GW3965 and T0901317 in the weaker induction of coactivator binding induced by the latter compounds. Importantly, the particular coactivator to corepressor ratio present in a cell, together with the competition for binding between coactivators and corepressors and the differential effects of LXR agonists on coactivator/corepressor recruitment, can explain the tissue-specific conduct of LXR agonists, providing novel elements to assist in the design of LXR agonists.

### Method of Obtaining Compound I: (22S,23S)-22,23-dihydroxystigmast-4-en-3-one

In a balloon provided with a refrigerant, in an inert atmosphere, 15 grams of stigmasterol in 750 ml of toluene anhydride are dissolved. 25 ml of N-methyl-4-piperidone are added; the mixture is stirred and boiled until 50 ml of solvent are distilled.

The mixture is cooled to 60° C., and 7 grams of aluminum isopropoxide are added. The solution is refluxed during 3 hours, and taken to ambient temperature and successively washed with 200 ml of 5% aqueous hydrochloric acid, 100 ml of aqueous sodium bicarbonate and finally water. Toluene is evaporated at reduced pressure and the resulting solid is recrystallized from methanol. 12.3 grams of (22E)-stigmast-4-en-3-one are obtained, melting point 127-128° C.

The product obtained is dissolved in a mixture consisting of 500 ml tetrahydrofuran and 100 ml water, and 1.5 grams of sodium bicarbonate, 10 mL tert-butanol, 2.8 grams of methanesulphonamide and 150 mg osmium tetroxide are added.

The resulting solution is heated to 50° C. during 24 hours and taken to ambient temperature. 12 grams of sodium bisulphate dissolved in 100 ml water are added. The volume of solvent is reduced to reduced pressure to about 300 mL. The mixture obtained is extracted 3 times with 100 mL of ethyl acetate. The organic extract is dried with sodium sulphate anhydrous and evaporated to dryness at reduced pressure.

The crude product is purified by silica column chromatography (eluting solvent:hexane/ethyl acetate 1:1). 8.9 grams of (22S,23S)-22,23-dihydroxystigmast-4-en-3-one are obtained.

¹H-RMN (CDCl₃, 200 MHz): 5.72 (1H, s, H-4); 3.61 (2H, m, H-22 and H-23).

¹³C-RMN (CDCl₃, 50 MHz): 198.4 (C-3); 170.4 (C-5); 123.9 (C-4); 72.3 (C-22); 70.7 (C-23).

IR: 3300 and 1680 cm⁻¹.

### Method of Obtaining Compound II: (22S,23S)-22,23-dihydroxystigmasta-1, 4-dien-3-one

120 mg of (22S,23S)-22,23-dihydroxystigmast-4-en-3-one are dissolved in 15 mL dioxane anhydrous. 180 mg of 2,3-dichlorine-5,6-diciano-1,4-benzoquinone (DDQ) are added and the mixture is refluxed, with stirring and inert atmosphere, during 24 hours.

The resulting suspension is filtrated and the filtrate evaporated to dryness. The resulting crude product is purified by silica column chromatography (eluting solvent: hexane/ethyl acetate 1:1). 87 mg of (22S,23S)-22,23-dihydroxystigmasta-1,4-dien-3-one are obtained.

¹H-RMN (CDCl₃, 200 MHz): 6.50 (1H, d, J=10 Hz, H-1); 5.93 (1H, d, J=10 Hz, H-2); 5.80 (1H, s, H-4); 3.61 (2H, m, H-22 and H-23)

¹³C-RMN (CDCl₃, 50 MHz): 186.0 (C-3); 168.4 (C-5); 155.3 (C-1); 127.4 (C-2); 123.8 (C-4); 72.3 (C-22); 70.7 (C-23). IR: 3300 and 1665 cm⁻¹

### Synthesis of the Fluorinated Analogs of Present Invention (Compounds III, IV, V and VI):

The synthesis of the fluorinated analogs is depicted in the following Scheme 1:

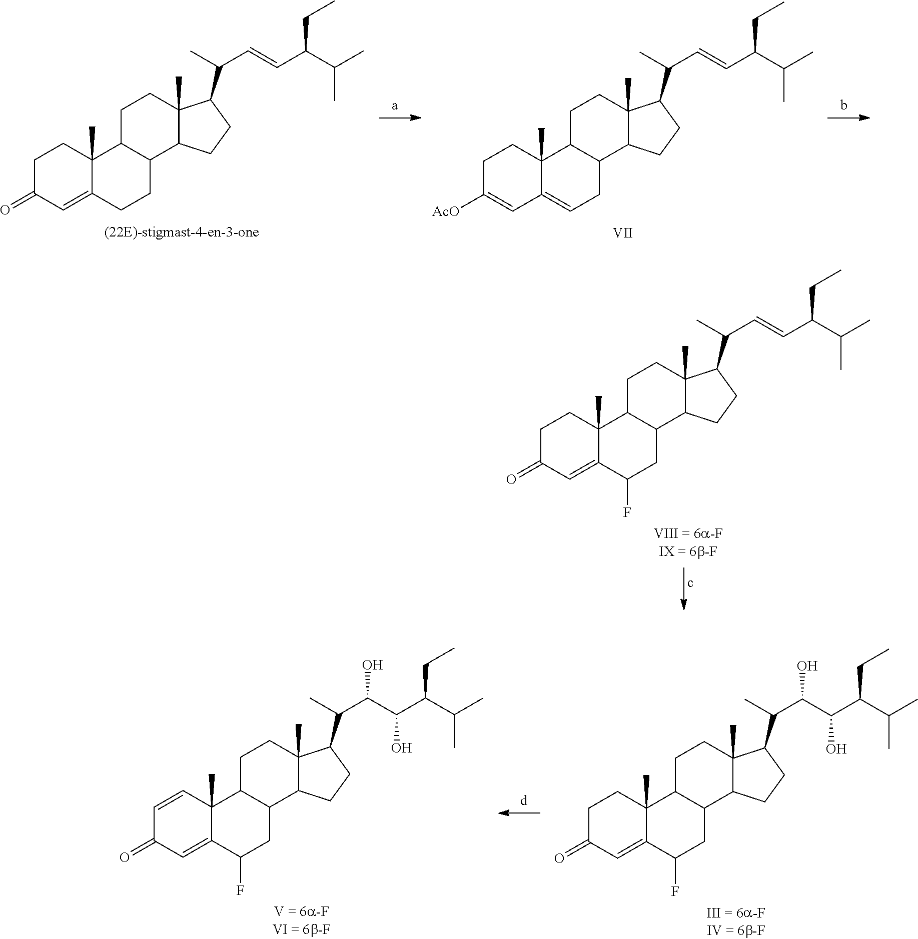

Fluorination at C-6 was achieved via a well-established procedure that signifies the electrophilic fluorination of the corresponding steroidal 3,5-dienol acetate. Thus, the dienol acetate 6, which was obtained from commercial stigmasterol in two steps, was subjected to fluorination with 1-(chloromethyl)-4-fluoro-1,4-diazabicyclo [2.2.2] octane-bistetrafluoroborate (Selectfluor). This fluorinating agent was chosen because it is the most suitable reagent for the required fluorination.

The mixture of the 6α- and 6β-fluoroenones 7a and 7b, which was obtained in a 3:1 ratio, was separated by column chromatography. The configuration assignment at C-6 was established from the coupling patterns observed in the ¹H-NMR spectra of the corresponding H-6. In compound 7a, besides the H—F coupling, the H-6 shows three additional H—H couplings, being the larger of 12.2 Hz. It suggests the presence of an axial-axial coupling for this proton, which is only compatible with a 6α configuration for the geminal fluorine. Furthermore, in compound 7b not only this coupling pattern is absent, but a small long-range coupling between fluorine and the methyl-19 is observed, a typical feature of 6β-fluorosteroids.

Subsequently, the Δ 22 double bond of the 6α-fluoro compound 7a was selectively dihydroxylated under Sharpless' conditions using (DHQ) 2-Phal as the catalyst and producing compound 3a as the only product. The outcome of the reaction was the desired 22S,23S diol, a moiety that, as our previous studies suggest, is essential for antiherpetic activity. The configuration of the diol was established by comparison with the chemical shifts and coupling constants of known closely related structures.

Finally, compound 3a was treated with 2,3-dichloro-5,6-dicyano-1,4-benzoquinone (DDQ) to introduce an additional Δ 1 double bond, affording compound 4a with 81% yield. Similarly, application of the dihydroxylation-dehydrogenation sequence on the 6β-fluoro isomer 7b yielded compounds 3b and 4b.

The compounds obtained above comprise the following:
I (22S,23S)-22,23-dihydroxystigmast-4-en-3-one
II (22S,23S)-22,23-dihydroxystigmasta-1,4-dien-3-one
III (22S,23S)-6α-fluoro-22,23-dihydroxystigmast-4-en-3-one
IV (22S,23S)-6β-fluoro-22,23-dihydroxystigmast-4-en-3-one
V (22S,23S)-6α-fluoro-22,23-dihydroxystigmasta-1,4-dien-3-one
VI (22S,23S)-6β-fluoro-22,23-dihydroxystigmasta-1,4-dien-3-one

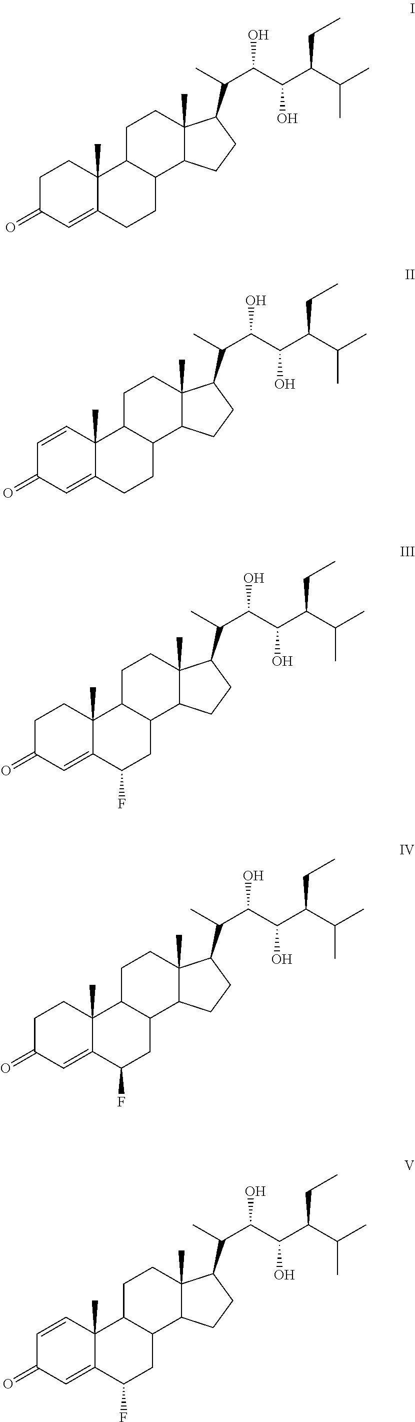 [SMILES: CC[C@@H](C(C)C)[C@H](O)[C@@H](O)C(C)[C@H]1CCC2C3CCC4=CC(=O)CC[C@]4(C)C3CC[C@@]21C ; CC[C@@H](C(C)C)[C@H](O)[C@@H](O)C(C)[C@H]1CCC2C3CCC4=CC(=O)C=C[C@]4(C)C3CC[C@@]21C ; CC[C@@H](C(C)C)[C@H](O)[C@@H](O)C(C)[C@H]1CCC2C3C[C@H](F)C4=CC(=O)CC[C@]4(C)C3CC[C@@]21C ; CC[C@@H](C(C)C)[C@H](O)[C@@H](O)C(C)[C@H]1CCC2C3C[C@@H](F)C4=CC(=O)CC[C@]4(C)C3CC[C@@]21C ; CC[C@@H](C(C)C)[C@H](O)[C@@H](O)C(C)[C@H]1CCC2C3C[C@H](F)C4=CC(=O)C=C[C@]4(C)C3CC[C@@]21C]

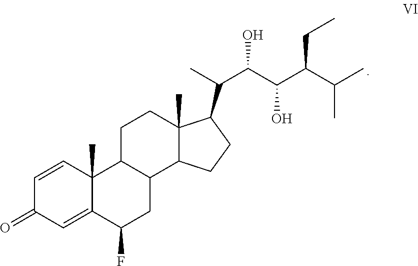 [SMILES: CC[C@@H](C(C)C)[C@H](O)[C@@H](O)C(C)[C@H]1CCC2C3C[C@@H](F)C4=CC(=O)C=C[C@]4(C)C3CC[C@@]21C]

### Compositions of Brassinosteroids of General Formula (a):

In one embodiment of the invention, a formulation for treating of skin diseases selected from the group consisting of psoriasis, photoaging, rosacea and UV induced skin cancer comprises (a) brassinosteroids of general formula (a)

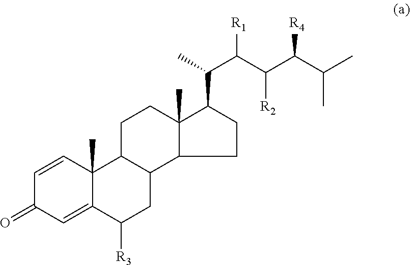

wherein,

R₁, R₂, and R₃ are selected from H, HO—, linear or branched C1-C4 alkyl, R₅—O—, HCOO—, R₅—COO—, —OOC—R₆—COO—, p-toluene sulphonate, phosphate, tartrate, maleate, sulphate, fluorine, chlorine, bromine, iodine and methanesulphonate,

R₄ and R₅ are selected from H and linear or branched C1-C4 alkyl,

R₆ is —(CH₂)ₙ— wherein n equals to 1, 2 or 3, and

[symbol] can be a single or double bond,

and a pharmaceutically acceptable additive, the pharmaceutically acceptable additive being a component selected from carrier, binding agent, stabilizer, adjuvant, diluent, excipient, surfactant, odorant, or dye.

In other embodiment of the invention, a formulation for treating of skin disease selected from the group consisting of of psoriasis, photoaging, rosacea and UV induced skin cancer comprises (22S,23S)-22,23-dihydroxystigmast-4-en-3-one (Compound I), and pharmaceutically acceptable additive, the pharmaceutically acceptable additive being a component selected from carrier, binding agent, stabilizer, adjuvant, diluent, excipient, surfactant, odorant, or dye. In other embodiment of the invention, a formulation for treating of skin disease selected from the group consisting of of psoriasis, photoaging, rosacea and UV induced skin cancer comprises comprises (22S,23S)-22,23-dihydroxystigmasta-1,4-dien-3-one (Compound II), and an additive, the additive being a component selected from carrier, binding agent, stabilizer, adjuvant, diluent, excipient, surfactant, odorant, or dye. A formulation according to the invention may further comprise a second pharmaceutically active agent selected from corticosteroids.

The composition according to the invention, comprising brassinosteroids of general formula (a) selected from
III (22S,23S)-6α-fluoro-22,23-dihydroxystigmast-4-en-3-one
IV (22S,23S)-6β-fluoro-22,23-dihydroxystigmast-4-en-3-one
V (22S,23S)-6α-fluoro-22,23-dihydroxystigmasta-1,4-dien-3-one
VI (22S,23S)-6β-fluoro-22,23-dihydroxystigmasta-1,4-dien-3-one as active ingredient, which is administered, in a preferred embodiment, orally, for example, as tablets or lozenges or capsules, in suspensions or emulsions, or in solutions, in powders or granules, or in syrups or elixirs. Compositions intended for oral use may be prepared according to any method known to the art for the manufacture of pharmaceutical compositions and such compositions may contain one or more agents selected from the group consisting of sweetening agents, flavoring agents, coloring agents and preserving agents in order to provide pharmaceutically acceptable preparations. The tablets may be uncoated or they may be coated by known techniques to delay disintegration and absorption. A composition comprising a brassinosteroid general formula (I) may be employed as a food additive. A low toxicity of brassinosteroids enables to employ safely sufficiently high therapeutic doses. For example, daily oral doses, for an adult subject, may comprise from about 10 ug to about 1000 mg of brassinosteroid of general formula (a).

In a method according to the invention, brassinosteroids general formula (I), may be administered orally or parenterally. For example, a composition comprising brassinosteroids general formula (a) may be administered intramuscularly, intraperitoneally, or intravenously. In one embodiment, the active formulation may be inserted to the body of a subject in need of the treatment by subcutaneous injection. On other embodiment, a deposit or an implant is inserted into the body, providing a slow release of brassinosteroids general formula (a) in the body.

The brassinosteroids (22S,23S)-22,23-dihydroxystigmast-4-en-3-one (Compound I), (22S,23S)-22,23-dihydroxystigmasta-1,4-dien-3-one (Compound II), (22S,23S)-6α-fluoro-22,23-dihydroxystigmast-4-en-3-one (Compound III), (22S,23S)-6β-fluoro-22,23-dihydroxystigmast-4-en-3-one (Compound IV), (22S,23S)-6α-fluoro-22,23-dihydroxystigmasta-1,4-dien-3-one (Compound V) and (22S,23S)-6β-fluoro-22,23-dihydroxystigmasta-1,4-dien-3-one (Compound VI), were formulated as the following composition:

| | | |
|---|---|---|
| Compound I, II, III, IV, V or VI: | 1 | mg |
| Sodium sulfate | 36.0 | mg |
| Sodiun Chloride | 9.0 | mg |
| EDTA | 0.3 | mg |
| Hydroxyethylcellulose | 10.0 | mg |
| Tyloxapol | 1.5 | mg |
| Distilled water | 3 | mL |

Compositions of the present invention further comprise corticosteroids including, but not limited to, hydrocortisone, triamcinolone, fluocinonide, betamethasone dipropionate, clobetasol, fluocinolone acetonide, prednisone, prenisolone, dexamethasone.

In a variant of the method of treatment proposed a composition comprising corticosteroids could be separately or sequentially administered in order to obtain the best improvement of the method of treatment for skin diseases. Prophylactic use of a compound of the invention can be conducted preceding the appearance of skin aging signs, in order to retard or prevent its advancement. Also, the compounds of the invention can be uses for therapeutic treatment of skin aging.

Systemic administration of the prophylactic or therapeutic compositions described herein can be conducted by the intravenous, subcutaneous, oral, intraperitoneal, intramuscular or transdermal routes, or any other suitable route. Also, the compounds of the invention can be topically administered in the form of a solution, a powder, an aerosol or a semi-solid composition. A semi-solid composition includes a jelly, an ointment, a cream, lotion, or other pharmaceutical presentations of considerably analogous density as to be applied to the skin.

The preparation of pharmaceutical carriers may include physiological saline solution, other non-toxic salts at physiological concentrations, sterile water, 5% aqueous glucose, as well as anti-fungal and anti-bacterial agents, dispersion media, and absorption delaying agents, among others. For ointments and creams thickening agents may include cetostearyl alcohol, propylene glycol, polyethylene glycols, aluminum stearate, hydrogenated lanolin, among others. The formulation of lotions may include dispersing agents, suspending agents, emulsifying agents, thickening agents, stabilizing agents, coloring agents or perfuming agents. Powders may be prepared by use of talc, starch, lactose, or other similar agents.

Transdermal delivery of the compounds of the invention can also be provided by dermal patches, which can include an absorption enhancer, for example DMSO. To attain beneficial pharmacodynamic or pharmacological effects, the compounds of the invention can be conjugated with other molecules such as polyethylene glycol. To achieve delivery of the compounds of the invention to the cell's cytosol, they can be conjugated with a carrier, including—but not limited to—a liposome. For topical applications, a permeation agent may include DMSO, decylmethyl sulfoxide, diethyleneglycolmonoethylether, cyclodextrins, pyrrolidones, urea derivatives and terpenes, among others.

### SUMMARY

We have shown that the compounds of the invention,
1—Activate LXR-receptors
2—Reduce the signs of psoriasis in an experimental model, showing protective effects that complement those exhibited by the currently used therapy, i.e., corticosteroids
3—In combination with corticosteroids provide better protection against mouse psoriatic skin lesions than treatment with clobetasol alone
4—Stimulate the expression of LXR-alpha and LXR-beta, and reduce the expression of TNF-alpha and IL-8 in a human keratinocyte cell line irradiated with UV (photoaging model)
5—Do not activate genes responsible for unwanted LXR effects in cultured cells, such hypertriglyceridemia and hepatic steatosis
6—Modulate the expression of LXR-dependent genes in a selectively way, and as a result they lack the in vivo adverse effects (hypertriglyceridemia and hepatic steatosis) of synthetic LXR agonists

We have shown that the compounds of the invention activate LXR-alpha receptors without the adverse effects of LXR agonists. Restoring LXR-alpha expression/function within a psoriatic lesion may contribute to reverse the transition from psoriatic to symptomless skin. The present invention proposes the combination of corticosteroids and brassinosteroid analogs as a means of attaining anti-psoriasis efficacy while reducing the unwanted effects associated to corticosteroid therapy, and without the adverse effects of LXR activators.

We propose that the compounds of the invention would improve rosacea symptoms, due to their ability to improve barrier function and modulate cytokine content through LXR activation.

Thus, the invention refers to a composition of topical and systemic use for treating psoriasis; in addition, due to the similarity between the underlying mechanisms in the pathogenesis of psoriasis and those involved in photoaging, rosacea and UV induced skin cancer, we propose the use of a topical/systemic composition including the compounds of the invention, alone or in combination with corticosteroids for the treatment of compounds of photoaging, rosacea and UV induced ski cancer.

1. A method of treatment of skin diseases comprising
administering to a patient in need thereof of a composition that comprises brassinosteroid analogs of general formula (a) 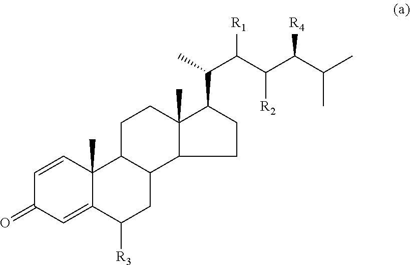
wherein,
R₁, R₂, and R₃ are selected from H, HO—, linear or branched C1-C4 alkyl, R₅—O—, HCOO—, R₅—COO—, —OOC—R₆—COO—, p-toluene sulphonate, phosphate, tartrate, maleate, sulphate, fluorine, chlorine, bromine, iodine and methanesulphonate,
R₄ and R₅ are selected from H and linear or branched C1-C4 alkyl,
R₆ is —(CH₂)ₙ— wherein n equals to 1, 2 or 3, and [symbol]
can be a single or double bond,
and a pharmacologically acceptable excipient.

2. The method of treatment of skin diseases according to claim 1, wherein the brassinosteroid analogs are selected from the group comprising:

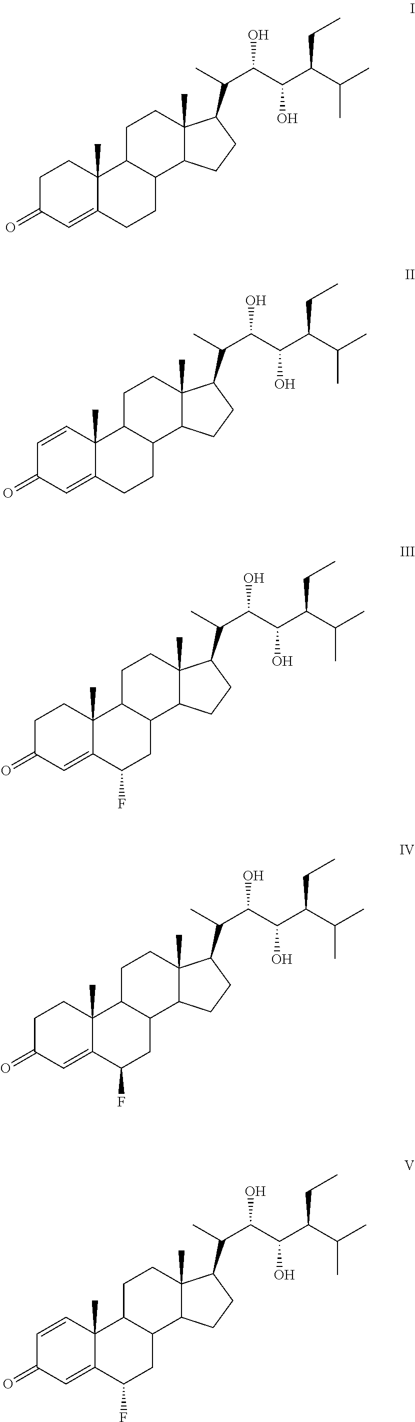 [SMILES: CC[C@@H](C(C)C)[C@H](O)[C@@H](O)C(C)[C@H]1CCC2C3CCC4=CC(=O)CC[C@]4(C)C3CC[C@@]21C ; CC[C@@H](C(C)C)[C@H](O)[C@@H](O)C(C)[C@H]1CCC2C3CCC4=CC(=O)C=C[C@]4(C)C3CC[C@@]21C ; CC[C@@H](C(C)C)[C@H](O)[C@@H](O)C(C)[C@H]1CCC2C3C[C@H](F)C4=CC(=O)CC[C@]4(C)C3CC[C@@]21C ; CC[C@@H](C(C)C)[C@H](O)[C@@H](O)C(C)[C@H]1CCC2C3C[C@@H](F)C4=CC(=O)CC[C@]4(C)C3CC[C@@]21C ; CC[C@@H](C(C)C)[C@H](O)[C@@H](O)C(C)[C@H]1CCC2C3C[C@H](F)C4=CC(=O)C=C[C@]4(C)C3CC[C@@]21C]

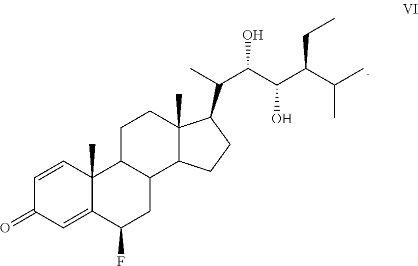 [SMILES: CC[C@@H](C(C)C)[C@H](O)[C@@H](O)C(C)[C@H]1CCC2C3C[C@@H](F)C4=CC(=O)C=C[C@]4(C)C3CC[C@@]21C]

3. The method of treatment of skin diseases according to claim 1, wherein the skin disease is selected from the group comprising psoriasis, skin aging, rosacea, dermatitis, burns, skin cancer and malignancies, and pigmentary derangements.

4. The method of treatment of skin diseases according to claim 3, wherein the skin aging includes chronological aging and UV-induced aging.

5. The method of treatment of skin diseases according to claim 3, wherein the pigmentary derangements include vitiligo.

6. The method of treatment of skin diseases according to claim 1, wherein the composition is a systemic or topical composition.

7. The method of treatment of skin diseases according to claim 6, wherein the composition further comprises corticosteroids.

8. The method of treatment of skin diseases according to claim 1, the composition is separately or sequentially administered with corticosteroids.

9. The method of treatment of skin diseases according to claim 7, wherein the corticosteroids are selected from the group comprising hydrocortisone, triamcinolone, fluocinonide, betamethasone dipropionate, clobetasol, fluocinolone acetonide, prednisone, prenisolone, dexamethasone.

10. A composition comprising brassinosteroid analogs of general formula (a) 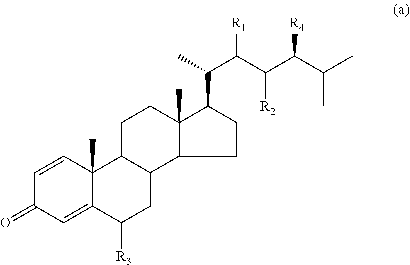
wherein,
R₁, R₂, and R₃ are selected from H, HO—, linear or branched C1-C4 alkyl, R₅—O—, HCOO—, R₅—COO—, —OOC—R₆—COO—, p-toluene sulphonate, phosphate, tartrate, maleate, sulphate, fluorine, chlorine, bromine, iodine and methanesulphonate,
R₄ and R₅ are selected from H and linear or branched C1-C4 alkyl,
R₆ is —(CH₂)ₙ— wherein n equals to 1, 2 or 3, and [symbol]
can be a single or double bond, and a pharmacologically acceptable excipient for use in the treatment of skin diseases.

11. The composition for use in the treatment of skin diseases according to claim 10, wherein the brassinosteroid analogs are selected from the group comprising:

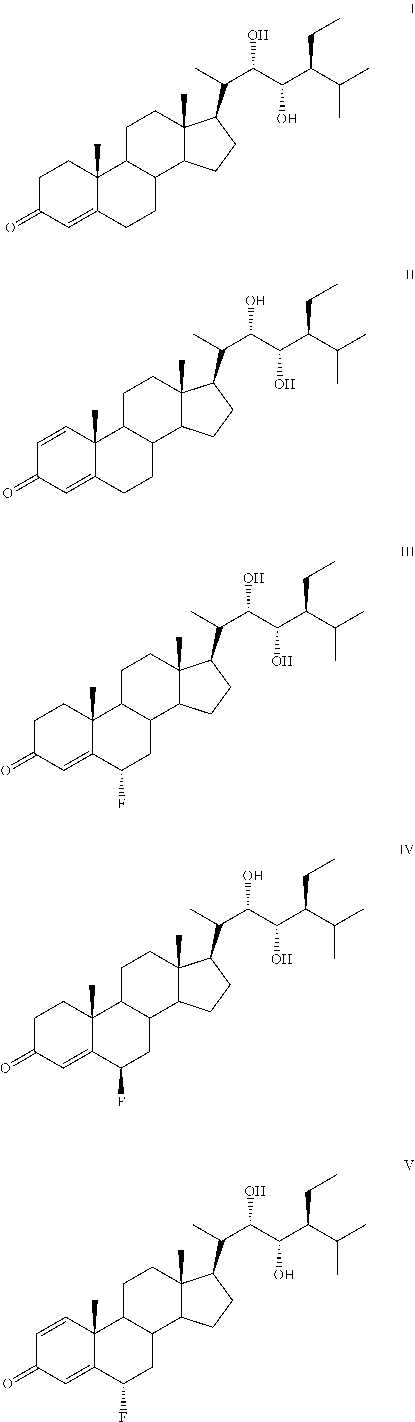 [SMILES: CC[C@@H](C(C)C)[C@H](O)[C@@H](O)C(C)[C@H]1CCC2C3CCC4=CC(=O)CC[C@]4(C)C3CC[C@@]21C ; CC[C@@H](C(C)C)[C@H](O)[C@@H](O)C(C)[C@H]1CCC2C3CCC4=CC(=O)C=C[C@]4(C)C3CC[C@@]21C ; CC[C@@H](C(C)C)[C@H](O)[C@@H](O)C(C)[C@H]1CCC2C3C[C@H](F)C4=CC(=O)CC[C@]4(C)C3CC[C@@]21C ; CC[C@@H](C(C)C)[C@H](O)[C@@H](O)C(C)[C@H]1CCC2C3C[C@@H](F)C4=CC(=O)CC[C@]4(C)C3CC[C@@]21C ; CC[C@@H](C(C)C)[C@H](O)[C@@H](O)C(C)[C@H]1CCC2C3C[C@H](F)C4=CC(=O)C=C[C@]4(C)C3CC[C@@]21C]

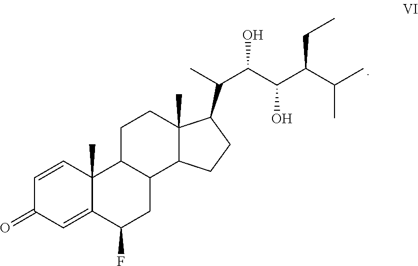 [SMILES: CC[C@@H](C(C)C)[C@H](O)[C@@H](O)C(C)[C@H]1CCC2C3C[C@@H](F)C4=CC(=O)C=C[C@]4(C)C3CC[C@@]21C]

12. The composition for use in the treatment of skin diseases according to claim 10, wherein the skin disease is selected from the group comprising psoriasis, skin aging, rosacea, dermatitis, burns, skin cancer and malignancies, and pigmentary derangements.

13. The composition for use in the treatment of skin diseases according to claim according 12, wherein the skin aging includes chronological aging and UV-induced aging.

14. The composition for use in the treatment of skin diseases according to claim according 12, wherein the pigmentary derangements include vitiligo.

15. The composition for use in the treatment of skin diseases according to claim according to claim 10, wherein the composition is a systemic or topical composition.

16. The composition for use in the treatment of skin diseases according to 15, wherein the composition further comprises corticosteroids.

17. The composition for use in the treatment of skin diseases according to claim 10, wherein the composition is separately or sequentially administered with corticosteroids.

18. The composition for use in the treatment of skin diseases according to claim 16, wherein the corticosteroids are selected from the group comprising hydrocortisone, triamcinolone, fluocinonide, betamethasone dipropionate, clobetasol, fluocinolone acetonide, prednisone, prenisolone, dexamethasone.

19. The method of treatment of skin diseases according to claim 2, wherein the skin disease is selected from the group comprising psoriasis, skin aging, rosacea, dermatitis, burns, skin cancer and malignancies, and pigmentary derangements.


SEQUENCE LISTING (14 sequences)

SEQ ID NO: 1 - DNA, 22 bp, Artificial Sequence
  ABCA1 Fw
gagtgaagcctgtcatctactg

SEQ ID NO: 2 - DNA, 22 bp, Artificial Sequence
  ABCA1 Bw
gagtgaagcctgtcatctactg

SEQ ID NO: 3 - DNA, 22 bp, Artificial Sequence
  ABCG1 Fw
tcctcttcaagaggaccttcct

SEQ ID NO: 4 - DNA, 18 bp, Artificial Sequence
  ABCG1 Bw
cccaatgtgcgaggtgat

SEQ ID NO: 5 - DNA, 21 bp, Artificial Sequence
  FAS fw
acagggacaacctggagttct

SEQ ID NO: 6 - DNA, 21 bp, Artificial Sequence
  FAS bw
ctgtggtcccacttgatgagt

SEQ ID NO: 7 - DNA, 20 bp, Artificial Sequence
  TNF-alpha Fw
ctgctgcactttggagtgat

SEQ ID NO: 8 - DNA, 19 bp, Artificial Sequence
  TNF-alpha Bw
acgctgcatagctcgttcc

SEQ ID NO: 9 - DNA, 20 bp, Artificial Sequence
  IL-8 Fw
ctgcgccaacacagaaatta

SEQ ID NO: 10 - DNA, 20 bp, Artificial Sequence
  IL-8 Bw
attgcatctggcaaccctac

SEQ ID NO: 11 - DNA, 22 bp, Artificial Sequence
  LXR-alpha Fw
ccttcagaacccacagagatcc

SEQ ID NO: 12 - DNA, 19 bp, Artificial Sequence
  LXR-alpha Bw
acgctgcatagctcgttcc

SEQ ID NO: 13 - DNA, 22 bp, Artificial Sequence
  LXR-beta Fw
tttgagggtatttgagtagcgg

SEQ ID NO: 14 - DNA, 19 bp, Artificial Sequence
  LXR-beta Bw
ctctcgcggagtgaactac